(19) United States
(12) Patent Application Publication
(10) Pub. No.: US 2012/0052082 A1
(43) Pub. Date: Mar. 1, 2012

(54) CROSS-PROTECTIVE INFLUENZA VACCINE

(75) Inventors: Richard W. Compans, Atlanta, GA (US); Sang-Moo Kang, Lilburn, GA (US); Jadranka Bozja, Tucker, GA (US); Bao-Zhong Wang, Duluth, GA (US); Jae-Min Song, Decatur, GA (US)

(73) Assignee: Zetra Biologicals, LLC

(21) Appl. No.: 13/084,367

(22) Filed: Apr. 11, 2011

Related U.S. Application Data
(60) Provisional application No. 61/322,713, filed on Apr. 9, 2010.

(51) Int. Cl. A61K 39/145; A61P 31/16; A61P 37/04

(52) U.S. Cl. 424/186.1; 424/192.1

(57) ABSTRACT

A cross-protective influenza virus vaccine has been designed based on the incorporation of the genetically engineered, highly conserved M2 influenza viral protein optionally in combination with an adjuvant such as a bacterial flagellin protein incorporated into the membrane of a virosome or virus-like particles. Immunogenicity and the breadth of cross protection efficacy are significantly enhanced using multiple copies of the influenza M2 protein as a membrane bound tetramer and/or in combination with a membrane bound adjuvant. A method for vaccinating a subject for influenza A has also been developed that results in broad and improved cross-protection against multiple subtypes of influenza A virus.

### CROSS REFERENCE TO RELATED APPLICATIONS

This application claims benefit under 35 U.S.C. 119 of U.S. Provisional Application No. 61/322,713, filed Apr. 9, 2010, which is hereby incorporated herein by reference in its entirety.

### STATEMENT REGARDING FEDERALLY SPONSORED RESEARCH OR DEVELOPMENT

This invention was made with Government Support under Agreement AI068003 awarded to Richard W. Compans by the National Institutes of Health. The Government has certain rights in the invention.

### REFERENCE TO SEQUENCE LISTING

The Sequence Listing being submitted herewith as a text file named “ZET_{—}100 ST25.txt,” created on Apr. 11, 2011, and having a size of 4 kilobytes is hereby incorporated by reference pursuant to 37 C.F.R. §1.52(e)(5).

### FIELD OF THE INVENTION

This application is generally in the field of cross-protective influenza virus vaccines.

### BACKGROUND OF THE INVENTION

Influenza is one of the most important viral diseases in humans, with significant medical and economic burdens. Typically 10-20% of the world population is infected during seasonal epidemics resulting in three to five million cases of severe illness and 250,000 to 500,000 lethal cases per year. In the US, influenza kills an average of 20,000-40,000 people per year, causes an average of over 100,000 influenza-related hospitalizations and results in an economic cost of $12 billion per year. Development of broadly protective vaccines against influenza virus has proven to be difficult due to the high mutation rate of the surface proteins. Moreover, novel influenza A strains containing new hemagglutinin (HA) and/or neuraminidase (NA) genes acquired from animal influenza viruses occasionally appear in the human population causing pandemics.

Influenza is an enveloped, single-stranded RNA virus of the orthomyxovirus family. Influenza viruses are typed as A, B or C based on the antigenic characteristics of the nucleoprotein (NP) and matrix (M) protein antigens. Subtypes of influenza A are determined by the surface antigens hemagglutinin (HA) and neuraminidase (NA). There are 16 recognized HA subtypes (H1-H16) and 9 recognized NA subtypes (N-1-N9). All of these subtypes have been isolated in birds, but only 3 different HA (H1, H2 and H3) and 2 different NA (N1 and N2) subtypes commonly circulate in humans. Only H1N1, H1N2, and H3N2 are currently in general circulation among people. Other subtypes are found most commonly in other animal species. For example, H5N1 is considered an avian disease; however, there is some evidence of limited human-to-human transmission of the virus, and H5N1 may mutate or reassort into a strain capable of efficient human-to-human transmission.

Once infected with an influenza virus, a person develops protective antibodies (predominantly HA/NA-specific) against the virus. Influenza viruses undergo changes over time, allowing them to evade the host immune system and to reduce the effectiveness of immunity to prior infections. Influenza A viruses can change by two different means: “antigenic drift” and “antigenic shift”. “Antigenic drift” results from point mutations in the HA and/or NA antigens that occur during viral replication and may render previous infection or vaccination with earlier virus strains unprotective against subsequent viruses. The regular recurrence of influenza epidemics is thought to be caused by antigenic drift, as a number of studies show that over some years sufficient changes accumulate in the virus to allow influenza to re-infect the same host. In contrast, antigenic shift involves major antigenic changes that can result in the appearance of new pandemic viruses, or new subtypes, which are introduced into a population that has no protective serum antibody. Each pandemic has been characterized by a major shift in one or both of the HA or NA antigens.

The currently used inactivated influenza vaccines induce immune responses to the viral surface glycoproteins HA and NA; although protective, the immunity is highly strain specific. Because these proteins undergo extensive antigenic variation, frequent changes are necessary in the vaccine composition. Although the current vaccines include proteins of the two currently circulating subtypes of influenza A viruses (H1N1 and H3N2), they are not effective in protecting against the spectrum of different antigenic subtypes of influenza A viruses that are abundant in avian species which could potentially cause new influenza pandemics in humans.

Available seasonal influenza vaccines are trivalent inactivated (killed) virus vaccines (TIV) or live, attenuated, trivalent influenza virus vaccine (LAIV). Type A and B strains of influenza virus contained in LAIV replicate in the nasal passages but not in the lower respiratory tract. These vaccines are targeted to primarily induce neutralizing antibodies directed against the viral envelope protein HA as well as the NA of a homologous strain to the virus used for vaccination. However, drifted strains can appear following annual formulation of the vaccine composition. Influenza virus strains that are not matched with the seasonal vaccine appear on a regular basis, which can have a significant impact on vaccine effectiveness. Such drifted strains can compromise vaccine-induced immunity due to antigenic mismatch with the vaccine strain, and resulting seroprotection rates can vary according to the antigenic distance between the vaccine strain and the circulating strain. Since 1972, WHO has recommended 39 changes in the influenza vaccine formulation. It has been suggested that approximately once every decade the mismatch between virus and vaccine is enough to reduce vaccine effectiveness by 70%. The major limitations of the current vaccines are the need to produce new vaccines every season, the uncertainty in choice of the correct strains, long period of vaccine production time as well as the fact that the vaccines are produced by a slow process requiring embryonated eggs. Improved vaccines are needed, not only for seasonal influenza, but also for a potential influenza pandemic. Therefore it is extremely valuable to develop new influenza vaccines targeted to more highly conserved antigens, which would have the potential to induce broad spectrum anti-influenza immunity.

It is therefore an object of the present invention to provide a cross-protective, strain non-specific and/or universal vaccine for influenza A viruses.

It is another object of the present invention to provide a method of vaccinating a subject that provides broad and improved cross-protection against multiple subtypes of influenza A viruses.

It is another object of the present invention to provide a method of making a vaccine which has advantages in terms of low cost of production, high potential for rapid distribution and delivery, and provides an alternative to delivery by injection.

### SUMMARY OF THE INVENTION

A cross-protective influenza vaccine has been developed. The basis of the vaccine is an influenza matrix protein 2 (M2) fusion protein immobilized on the surface of a virus-like particle (VLP). In some embodiments, the M2 fusion protein contains two or more influenza matrix protein 2 (M2) extracellular domains (M2e) and at least one adjuvant protein. The M2 fusion protein preferably contains at least four M2 extracellular domains (4.M2e), e.g., in tandem. In some embodiments, all of the M2 extracellular domains in the fusion protein are from the same strain, e.g., are identical. In other embodiments, the fusion protein contains M2 extracellular domains from at least two different strains or subtypes of influenza virus. For example, at least one of the M2 extracellular domains can have the amino acid sequence SEQ ID NO:2. The two or more M2 extracellular domains can also be separated by amino acid linkers, such as flexible linkers. Therefore, in some embodiments, the fusion protein contains the amino acid sequence SEQ ID NO:3.

Activation of the innate immune system through Toll-like receptors (TLR) is an effective-way to prime the immune system to activate strong adaptive immune responses. Once activated, TLR-expressing cells activate multiple arms of the immune system including anti-microbial effector molecules, type I and type II interferons, cytokines, chemokines, costimulatory molecules, and effective T and B cell priming by antigen presenting cells (APCs). Gram-negative flagellin is a Toll-like receptor 5 (TLR5) agonist and an effective adjuvant. Therefore, in some embodiments, the adjuvant protein in the M2 fusion protein is a bacterial flagellin protein. Flagellin phase-1 antigen from Salmonella (called FliC), the monomeric subunit protein which polymerizes to form the filaments of bacterial flagella, is a polypeptide without cystine residues and limited post-translational modification of lysine residues. Therefore, in some embodiments, the adjuvant protein in the M2 fusion protein is FliC.

Native M2 protein assembles into a tetramer in the host membrane during viral uncoating and maturation. Therefore, in preferred embodiments, the M2 fusion protein contains an oligomerization domain, such as a tetramerization domain. The tetramerization domain can be a natural or engineered tetramerization domain. For example, the tetramerization domain can have the amino acid sequence SEQ ID NO:4.

In some embodiments, the fusion protein includes a signal peptide to facilitate secretion of the VLP. In some embodiments, the fusion protein contains a membrane anchor to immobilize the fusion protein on the surface of the VLP. For example, the membrane anchor can include a transmembrane domain and optionally a cytoplasmic domain of a viral envelope protein. In some embodiments, the fusion protein contains the transmembrane domain and optionally the cytoplasmic domain of wild-type M2, or a conservative variant and/or fragment thereof that intercalates into the VLP membrane. In other embodiments, the fusion protein contains the transmembrane domain and optionally the cytoplasmic domain of hemagglutinin (HA), neuraminidase (NA) protein, or a conservative variant and/or fragment thereof that intercalates into the VLP membrane.

VLPs are assembled when envelope proteins are co-expressed in a cell with viral core/matrix proteins, such as influenza virus matrix protein 1 (M1). The disclosed VLPs preferably contain M1, i.e., are M1 VLPs. Other matrix proteins such as VSV M, paramyxovirus M, HIV Gag, MMTV core protein, or lassa virus Z core protein can be used. Methods to enhance the incorporation of wild type M2 or tandem repeats of M2 ectodomain into VLPs are described, for example, by replacement of transmembrane and/or cytoplasmic domains of wild type M2 or tandem repeats of M2 ectodomain of M2 with sequences of other proteins known to be localized in cholesterol-rich lipid raft domains on cell surfaces, such as those from influenza hemagglutinin; replacement of transmembrane of wild type M2 or tandem repeats of M2 ectodomain of M2 with HIV Vpu* transmembrane domain (*Vpu transmembrane domain has the following sequence: MQPIQIAIVALVVAIIIAIVVWSIVII); anchoring the wild type M2 or tandem repeats of M2 ectodomains to the HA2 domain of influenza hemagglutinin including transmembrane and cytoplasmic domains (the HA2 domain of influenza hemagglutinin also has highly conserved fusion peptides); replacement of transmembrane and cytoplasmic domains of wild type M2 or tandem repeats of M2 ectodomain of M2 with those from MMTV Env protein; and any combinations of the above.

The disclosed VLP provides cross-protection by presenting an immunogenic amount and form of M2 protein on the surface of the VLP. Therefore, in preferred embodiments, the M1 protein is the predominant or only protein on the surface of the VLP. For example, in some embodiments, the disclosed VLP does not contain HA or NA protein on the surface of the VLP.

Unexpected advantages have been obtained when the 4M2e/flagellin fusion protein is incorporated into virus like particles (“VLPs”) formed from M1 protein core but lacking HA and NA. Inoculation with inactivated viral vaccine supplemented with M2 VLPs provided complete cross protection. Expression of M2 constructs on VLPs in a membrane-anchored form more likely mimics the natural conformation on influenza viruses. Conjugation of M2e to the tetremeric oligomerization domain is more immunogenic and cross protective. In one embodiment, a phase I flagllin FliC is used in M2e-FliC VLPs. In most isolates of Salmonella, two genes encode flagellar antigens. FliC encodes the phase I flagellin FliC, and fljB encodes the phase II flagellin FljB. These genes are coordinately expressed by a phase-variation mechanism. Only one gene is expressed in a bacterium in a specific time. FlicC can be more universal since the majority is the FliC. In another embodiment, a modified second generation flagellin that has a deletion in the variable region known to elicit inflammatory responses has been used.

A method is also provided for vaccinating a subject for influenza A by administering to the subject the disclosed cross-protective influenza vaccine. For example, the vaccine can be administered intramuscularly, intranasally, or by microneedle to an individual in need thereof.

In addition, compositions and methods are provided for vaccinating a subject for influenza A that results in broad and improved cross protection against multiple subtypes of influenza A virus. The method involves administering to the subject inactivated or attenuated influenza A virus supplemented with a virus-like particle (VLP) assembled from recombinant influenza matrix protein 1 (M1) and matrix protein 2 (M2). Preferably, the VLP does not contain HA or NA. Therefore a composition is provided that contains therapeutically effective amounts of the inactivated or attenuated influenza A virus and the VLP for co-administration. In some embodiments, the inactivated or attenuated influenza A virus and the VLP are mixed in a single container. In other embodiments, the inactivated or attenuated influenza A virus and the VLP are in separate containers in a device or kit for use in co-administration.

Two types of seasonal influenza vaccine are licensed by the Food and Drug Administration (FDA) for use in the United States: trivalent influenza vaccine (TIV) and live, attenuated influenza vaccine (LAIV). In some embodiments, the inactivated influenza A virus is a TIV. Non-limiting examples of TIVs include AGRIFLU (Novartis), FLUZONE (Sanofi Pasteur), AFLURIA (CS Limited), FLULAVAL (ID Biomedical Corporation of Quebec), FLUARIX (GlaxoSmithKline), and FLUVIRIN (Novartis). In some embodiments, the attenuated influenza A virus a LAIV. For example, the LAIV can be FLUMIST (MedImmune Vaccines).

In some embodiments, VLPs assembled from M1 and M2 protein is administered to the subject simultaneously with inactivated or attenuated influenza A virus. In some embodiments, compositions containing VLPs assembled from M1 and M2 protein are administered before or after the inactivated or attenuated influenza A virus. The preferred time periods would be 2 to 3 weeks after vaccination with inactivated influenza viruses. A range of dosage amounts for the M2 VLP supplementation would be 2 to 20 micro-grams in terms of M2 proteins incorporated into VLPs. The method can further involve vaccinating the subjects with booster doses of VLPs and/or inactivated or attenuated influenza A at least 1 week to 1 year after the initial inoculation with VLPs and inactivated or attenuated influenza A virus. The preferred time periods for boost immunizations would be 2 to 28 weeks after priming. Booster immunizations could be done with M2 VLP vaccine supplement alone or together with influenza vaccines together.

In preferred embodiments, the compositions containing VLPs are administered via the same route as the inactivated or attenuated influenza A virus. For example, in some embodiments, both VLPs and LAIV vaccines are administered intranasally. Similarly, in some embodiments, both VLPs and TIV vaccines are administered by injection. However, in other embodiments, the VLPs are administered by a different route and/or dosage form that the inactivated or attenuated influenza A virus.

In some embodiments, the VLP are produced by co-infecting cells (e.g., insect cells) with one or more recombinant vectors (e.g., baculoviruses) expressing the M1 and M2 proteins, culturing the cells under physiological conditions, and purifying VLPs from cell culture supernatants.

In some embodiments, the M2 protein is full-length M2 protein. In some embodiments, however, the M2 protein is a fusion protein containing at least one extracellular domain of an influenza M2 protein (M2e). For example, the fusion protein can contain at least four extracellular domains of influenza M2 (4.M2e). In some embodiments, all of the M2 extracellular domains in the fusion protein are identical (i.e., are homogenous). In other embodiments, the fusion protein contains M2 extracellular domains from at least two different strains or subtypes of influenza virus (i.e., are heterogeneous). For example, at least one of the M2 extracellular domains can have the amino acid sequence SEQ ID NO:2. The M2 extracellular domains can also be separated by amino acid linkers, such as flexible linkers. Therefore, the fusion protein can contain the amino acid sequence SEQ ID NO:3.

The fusion protein or VLP can further contain an adjuvant protein, such as a bacterial flagellin or pilli protein present on the surface of the VLP. In some embodiments, the flagellin is a membrane-anchored form of flagellin. In other embodiments, a peptide from flagellin is incorporated into a fusion protein with M2e (e.g., M2e). Also disclosed is a pharmaceutical composition containing the VLP and an adjuvant, such as alum.

### BRIEF DESCRIPTION OF THE DRAWINGS

FIGS. 1A-E are schematics showing the domain structures (FIGS. 1A-1D) of M2e constructs (FIG. 1E) used in the experiments described in the Examples. FIG. 1F is a schematic diagram of His-tagged 4.M2e, His-tagged 4.M2e-tFliC fusion protein, 4.M2e-tFliC fusion protein with a MMTV or HA membrane-anchor and tetrameric M2e stabilized by the GCN4 tetramerization repeats with a HA or MMTV membrane anchor. In the membrane-anchored tetrameric M2e, single M2e can also be presented as tandem repeats (4.M2e).

FIG. 2 is a bar graph showing serum 4.M2e-specific IgG endpoint titers from mice immunized with 10 μg of 4.M2e protein, 10 μg of 4.M2e-tFliC fusion protein, 50 μg of 4.M2e-tFliC-TM.CT_{MMTV}/M1 virus-like particles (VLPs), or a mixture of 10 μg of 4.M2e protein with 10 μg of tFliC/M1. Mice were immunize either intramuscularly (IM), intranasally (IN) or by microneedle (MN), as indicated. Six mice were immunized per group, 3 times each at 4 week intervals. Serum samples were collected after each immunization. Serum IgG endpoint titers were determined by ELISA using assay plates coated with 100 μl/well of recombinant tandem M2e (5 μg/μl). Immune serum samples were 2×stepwise diluted starting at 100×. Diluted samples were applied to plates for antibody-binding. Bound antibodies were detected by binding horseradish peroxidase (HRP)-goat anti-mouse IgG antibody and color was developed with tetramethylbenzidine (TMB) substrate. The greatest dilution which presented an OD450 2× higher than that of the negative control (naïve group) was designated as the endpoint titer.

FIG. 3 is a bar graph showing serum M2-specific IgG endpoint titers from mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1. Mice were immunize either intramuscularly (IM), intranasally (IN) or by microneedle (MN), as indicated. Six mice were immunized per group, 3 times each at 4 week intervals. Serum samples were collected after each immunization. Serum IgG endpoint titers were determined by cell surface ELISA using MDCK cells infected with A/PR8 at an MOI of 1. The MDCK cells were washed with PBS and fixed with 10% formalin at 12 hours post-infection. Bound antibodies were detected by binding HRP-goat anti-mouse IgG antibody and color was developed with TMB substrate.

FIG. 4 is a bar graph showing lung extract IgA endpoint titers from mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1. Mice were immunize either intramuscularly (IM), intranasally (IN) or by microneedle (MN), as indicated. Three mice were immunized per group, 3 times each at 4 week intervals. Lungs from immunized mice were collected and extracted with 1 ml PBS containing 0.05% Tween 20 per lung twice. Lung extract IgA endpoint titers were determined by ELISA using assay plates coated with 100 μl/well of recombinant tandem M2e (5 μg/μl). Immune serum samples were 2×stepwise diluted, and 100 diluted samples were applied to plates for antibody-binding. Bound antibodies were detected by HRP-goat anti-mouse IgA antibody and color was developed with TMB substrate. The greatest dilution which presented an OD450 2× higher than that of the negative control (naïve group) was designated as the endpoint titer.

FIG. 5 is a line graph showing change in body weight (% of pre-challenge) as a function of time (days) in naive mice or mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1, and subsequently infected with 5×LD₅₀ A/PR8. Mice were immunize either intramuscularly (IM), intranasally (IN) or by microneedle (MN), as indicated.

FIG. 6 is a line graph showing survival of mice (%) as a function of time (days) in naive mice or mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1, and subsequently infected with 5×LD₅₀ A/PR8. Mice were immunize either intramuscularly (IM), intranasally (IN) or by microneedle (MN), as indicated.

FIG. 7 is a line graph showing change in body weight (% of pre-challenge) as a function of time (days) in naive mice or mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1, and subsequently infected with 5×LD₅₀ A/Philippines. Mice were immunize either intramuscularly (IM), intranasally (IN) or by microneedle (MN), as indicated.

FIG. 8 is a line graph showing survival of mice (%) as a function of time (days) in naive mice or mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1, and subsequently infected with 5×LD₅₀ A/Philippines. Mice were immunize either intramuscularly (IM), intranasally (IN) or by microneedle (MN), as indicated.

FIG. 9 is a bar graph showing the effect of Alum as a mucosal adjuvant on inducing the heterosubtypic cross protection. Groups of mice (n=6) were intranasally immunized with M2 VLPs (10 μg, M2 VLP) or a mixture of M2 VLPs (10 μg) plus Alum (50 μg) (M2 VLP.Alum) at weeks 0 and 4. Immunized and naïve (unimmunized) mice were heterosubtypically challenged with A/Philippines virus (5 LD₅₀) at week 5 after the boost immunization.

FIG. 10 is a bar graph showing M2-specific immune responses after immunization with H1 VLP (Influenza VLPs (10 μg) containing A/PR8 HA (without M2)), PR8i (inactivated A/PR/8/34 virus (10 μg)), M2 VLPs (Influenza VLPs containing M2 (10 μg)), or M2.V+PR8i (M2 VLP (10 μg)+PR8i (10 μg)). All mice immunized with 10 μg vaccine in terms of total protein (n=6).

FIG. 11 is a line graph showing the effects of M2 VLPs on enhancing the cross protection against A/Philippines/82 virus. Groups of mice were intranasally immunized with inactivated A/PR8 virus (5 μg) alone or a mixture of M2 VLPs (10 μg) and inactivated A/PR8 virus (5 μg) at weeks 0 and 4. Naïve and immunized mice were challenged with a high lethal dose (12 LD₅₀) of A/Philippines virus. The body weight changes were daily monitored and recorded.

FIG. 12 is a line graph showing the effects of M2 VLPs on enhancing the cross protection (% body weight) against the 2009 pandemic A/California/2009 virus as a function of time (days post challenge). The group of mice immunized with inactivated vaccine supplemented with M2 VLPs demonstrated improved protections as shown by a significantly less loss in body weight.

FIG. 13A is a schematic diagram of wild type M2. EX, external domain (1-24 aa); TM, transmembrane domain (25-43 aa); CT, cytoplasmic domain (44-97 aa). FIGS. 13B and 13C are bar graphs showing the titer (×10⁴) M2e-specific IgG antibody (FIG. 13B) and H5N1 A/Vietnam/1203/04-specific IgG1 and IgG2a isotype antibodies (FIG. 13C) in mice intranasally challenged with a lethal dose (6×LD₅₀) of A/Phil (H3N2) virus at 4 wk after boost (n=9). PR8i, inactivated virus alone; PR8i+M2VLP, M2 VLP supplemented PR8i vaccine; PR8i+HIV VLP, HIV VLP supplemented PR8i vaccine; M2VLP, M2 VLPs only; control, M1 only VLPs.

FIG. 14A is a line graph showing weight change (%) in mice vaccinated with PR8i, control, M2VLP, PR8i+HIVVLP, or PR8i+M2VLP as a function of time (days) post challenge with heterosubtypic H3N2 virus. FIG. 14B is a bar graph showing lung viral titers (log₁₀ pfu/ml) determined by a plaque assay at day 4 after challenge (n=4 of 9 challenged mice) for mice vaccinated with PR8i, PR8i+M2VLP, or control. Asterisk indicates significant difference between PR8i and PR8i+M2VLP groups (**P<0.01).

FIG. 15A is a line graph showing weight change (%) in mice vaccinated with PR8i, PR8i+M2VLP, or control as a function of time (days) post challenge with a lethal dose (6×LD₅₀) of reassortant A/Vietnam/1203/04 (H5N1) or A/California/04/2009 (H1N1). FIG. 15B is a line graph showing weight change (%) in mice vaccinated with PR8i, PR8i+M2VLP, or control as a function of time (days) post challenge with influenza viruses 4 wks after boost (n=6) vaccination. FIG. 15C is a line graph showing weight change (%) in mice vaccinated with PR8i, PR8i+M2VLP, or control as a function of time (days) post intranasal challenged with a lethal dose (6×LD₅₀) of A/Phil (H3N2) virus (n=9) 7 mo after vaccination. Body weight changes were recorded daily. PR8i, vaccination with inactivated A/PR8 vaccine alone; PR8i+M2 VLP, inactivated A/PR8 vaccine supplemented with M2 VLPs; control, M1 only VLPs without M2. Bars indicate SDs.

FIG. 16A is a line graph showing weight change (%) in mice vaccinated with PR8i (1× or 2×) or PR8i+M2VLP (1×, 2×, 4×, or 8×) as a function of time (days) post challenge with a lethal dose (6×LD₅₀) of A/Phil (H3N2). Dilutions (in fold) of immune sera are indicated in parentheses. FIGS. 16B and 16C are line graphs showing weight change (%) (FIG. 16B) and survival rate (FIG. 16C) in naïve mice or mice vaccinated with PR8i+M2VLP/CL that were either not pretreated or were pretreated with clodronate liposomes (CL(+)) as a function of time (days) post challenge with a lethal dose (6×LD₅₀) of A/California/04/2009 (H1N1) (n=6, BALB/c mice). PR8i+M2, untreated naive mice with PR8i+M2VLP immune sera and virus; PR8i+M2/CL(+), Clodronate liposome-treated naive mice with PR8i+M2VLP immune sera and virus, naive, untreated naive mice with control immune sera and virus; naive/CL(+), clodronate liposome-treated naive mice with mock immune sera and virus.

FIG. 17 is a bar graph showing control and M2e specific IFN-γ-secreting cell responses (IFN-γ spots/10⁶ cells) from a cytokine ELISPOT assay using splenocytes from mice intranasally immunized with PR8i, PR8i+M2VLP, or control and isolated 4 d after challenge (n=4 challenged mice). Asterisk indicates a significant difference between PR8i and PR8i+M2VLP groups (***P<0.001).

FIG. 18A is a bar graph showing the titer of antibodies specific for M2e in mice (n=6) challenged with PR8i virus at 3 wk after boost vaccination with inactivated vaccine (PR8i, 2 μg) supplemented with 0, 1, 5, or 10 μg M2 VLPs (PR8i+M2VLP) or human immunodeficiency VLPs (PR8i+HIVVLP). Titers of antibodies specific to M2 were determined by ELISA using the extracellular M2 peptide as a coating antigen. FIGS. 18B to 18E are bar graphs showing the titer of antibodies specific for H5HA (FIG. 18B), M1 (FIG. 18C), A/Vietnam/1203/04 (reassortant H5N1) (FIG. 18D), and A/Phil (H3N2) (FIG. 18E) in mice (n=9) challenged with control or PR8i virus at 3 wk after prime and boost vaccination with inactivated vaccine (PR8i, 2 μg) supplemented with M2 VLPs (PR8i+M2VLP). FIG. 18F is a bar graph showing the titer of IgG1 and IgG2a isotype antibodies specific for H5HA in mice (n=9) challenged with control or PR8i virus at 3 wk after boost vaccination with inactivated vaccine (PR8i, 2 μg) supplemented with M2 VLPs (PR8i+M2VLP). PR8i, inactivated A/PR/8/34 virus alone; PR8i+M2VLP, inactivated PR8i supplemented with M2 VLPs (10 μg); control, VLP with M1 but not M2. Asterisk indicates a significant difference between PR8i and PR8i+M2VLP groups (*P<0.05, **P<0.01).

FIG. 19 is a bar graph showing HAI titers against homologous (H1N1 A/PR8), heterologous (H1N1 A/WSN), and heterosubtypic (H5N1 A/Vietnam, H3N2 A/Phil) viruses in mice (n=6) challenged with PR8i virus and vaccinated with inactivated vaccine (PR8i), PR8i supplemented with M2 VLPs (PR8i+M2VLP), or control. Titers of antibodies specific to M2 were determined by ELISA using the extracellular M2 peptide as a coating antigen. PR8i, immune sera from PR8i group; PR8i+M2VLP, immune sera from PR8i+M2VLP group; control, sera from control group.

FIG. 20A-20B are line graphs showing M2 VLP-supplemented vaccination does not induce heterosubtypic virus neutralization or growth inhibition activities. Influenza A/Phil (H3N2) virus (0.1 multiplicity of infection) was used to infect MDCK cells in the presence of serially diluted immune sera. Neutralizing activity was determined by conventional plaque formation assay using serially diluted mouse immune sera from groups of mice (FIG. 20A). Virus growth was monitored by a hemagglutination activity assay (FIG. 20B).

FIG. 21 is a line graph showing weight change (%) in mice vaccinated with heat-inactivated (56° C., 30 min) PR8i, heat-inactivated PR8i+M2VLP, or control as a function of time (days) post challenge with a lethal dose (6×LD₅₀) of A/Phil (H3N2). Daily changes in body weight are shown. PR8i, immune sera from PR8i group; PR8i+M2VLP, immune sera from

### DETAILED DESCRIPTION OF THE INVENTION

### I. Definitions

The term influenza “type” refers to the classification of influenza virus genera in the family Orthomyxoviridae based on the antigenic characteristics of the nucleoprotein (NP) and matrix (M) protein antigens. Influenza viruses types include influenza A, influenza B, and influenza C.

The term influenza “subtype” refer to the classification of influenza A viruses by antibody responses to influenza hemagglutinin (HA) and neuraminidase (NA). There are at least 16 different HA subtypes and 9 different NA subtypes, so many different combinations of HA and NA proteins are possible. Subtypes of influenza A virus are named according to their HA and NA surface proteins. Exemplary influenza subtypes include H1N1, H1N2, and H3N2.

The term influenza “strain” refers to genetic differences within a single influenza subtype. Often, these new strains appear when an existing virus spreads to humans from other animal species, or when an existing human strain picks up new genes from a virus that usually infects birds or pigs. Consequently, vaccination against one strain within a subtype may not confer protection against a new strain within the same subtype.

The term “cross-protective” refers to a vaccine that confers immunity to viral strains, subtypes, and/or types other than the one from which the vaccine components are derived. Therefore, a cross-protective vaccine can confer immunity to 2, 3, 4, 5 or more influenza strains, subtypes, and/or types.

The term “strain non-specific vaccine” refers to a cross-protective vaccine that immunizes a subject to all, or substantially all, strains of a virus subtype. Therefore, a strain non-specific vaccine can continue to be used to immunize subjects to a subtype of influenza for at least 10, 15, 20, 25, 30 years.

The term “pan” or “universal” vaccine refers to a vaccine that immunizes a subject to all, or substantially all, strains and subtypes of a virus type, such as influenza A.

The term “virus-like particle” or “VLP” refers to particles that are assembled from viral structural (e.g., envelope and matrix/core) proteins but do not contain viral genetic material.

The term “virosome” refers to a liposome containing viral envelope proteins embedded in the lipid membrane. Virosomes, unlike VLPs, are formed in vitro and do not contain viral core proteins. Generally, virosomes are spherical, unilamellar vesicles with a mean diameter of about 150 nm. In contrast to liposomes, virosomes generally contain functional viral envelope glycoproteins, such as influenza virus hemagglutinin (HA), neuraminidase (NA), or matrix protein 2 (M2), intercalated in the phospholipid bilayer membrane.

The term “reassortant virus” refers to a virion containing deoxyribonucleic acid from one virus species and a protein coat from another.

The term “immunogenic” refers to the ability of a substance, such as a peptide, to provoke an immune response in the body of a human or animal. For example, an immunogenic peptide can in some embodiments provoke specific antibody production or cellular immunity to the peptide when injected alone or in combination with other agents into a subject.

The term “subject” refers to any individual who is the target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes humans and animals.

The term “therapeutically effective amount” as used herein refers to an amount sufficient to produce in a subject a detectable increase in humoral and/or cellular immune response to one or more viruses.

“Simultaneous administration” includes 1) administration of a single composition containing both the VLPs and inactivated or attenuated influenza A virus; and 2) administration of a first composition containing the VLPs and a second composition containing inactivated or attenuated influenza A virus within 5, 10, 15, 30, or 60 minutes of each other.

The term “peptide” may be used to refer to a natural or synthetic molecule having two or more amino acids linked by the carboxyl group of one amino acid to the alpha amino group of another. The peptide is not limited by length; thus “peptide” can include polypeptides and proteins.

The term “protein domain” refers to a part of protein sequence and structure that can evolve, function, and exist independently of the rest of the protein chain. Because they are independently stable, domains can be “swapped” by genetic engineering between one protein and another to make chimeric proteins. For example, influenza M2 protein may be divided into three domains which include an extracellular domain (comprising 24 residues, M2e), a transmembrane domain (19 residues), and a cytoplasmic domain (54 residues).

A “chimeric molecule” is a single molecule created by joining two or more molecules that exist separately in their native state. The single, chimeric molecule preferably has the desired functionality of all of its constituent molecules.

A “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “spacer” or “linker” refers to a peptide that joins polypeptides within a fusion protein. Generally a linker has no specific biological activity other than to join the proteins or to preserve some minimum distance or other spatial relationship between them. However, the constituent amino acids of a linker may be selected to influence some property of the molecule such as the folding, net charge, or hydrophobicity of the molecule. For example, a “flexible linker” allows the connecting polypeptides to freely twist and rotate through space.

The term “signal peptide” refers to a peptide chain (e.g., 3-60 amino acids long) that directs the transport of a protein (e.g., to the outer membrane). Signal peptides may also be called targeting signals, signal sequences, transit peptides, or localization signals.

The term “adjuvant molecule” is an agent that can stimulate the immune system's response to a vaccine, without having any specific antigenic effect in itself. In particular the term includes bacterial surface proteins capable of targeting a host Toll-like receptor (TLR) protein, such as, but not limited to, a bacterial flagellin protein that targets a host TLR5 protein.

By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained.

### II. Viral Formulations

Because of limitations of current vaccines, a cross-protective, strain-non-specific, and/or universal vaccine that is based on the relatively conserved domains of the influenza virus is a promising approach. A precondition is the accessibility of these domains to antibodies on infectious virus particles, intact infected cells, or both. The extracellular domain of the influenza M2 protein (M2e) is highly conserved in influenza A viruses, and is an attractive antigen for such influenza vaccines if highly immunogenic candidates can be generated. In the virion, M2 immunogenicity is low because it is a small protein potentially masked by the major surface glycoproteins, and because it is presented in low amounts.

Compositions and methods overcome this limitation by incorporating the M2 protein into virus-like particles (VLPs). In preferred embodiments, the VLPs lack HA and NA so that the M2 protein is not masked.

In some embodiments the M2 protein is a protein fragment or fusion protein containing at least the M2e domain. In order to increase the immunogenicity of this peptide, the M2 protein can be multimeric (e.g., tetrameric). Their immunogenicity can be further enhanced by incorporation of an adjuvant such as a modified membrane-anchored form of flagellin, the natural ligand of the toll-like receptor 5 (TLR5). The resulting VLPs can elicit high titers of M2-specific antibodies and thereby confer protection against infection by a range of influenza A viruses.

Moreover, VLPs having M2 protein immobilized on the surface can be used to supplement existing influenza virus vaccines, such as inactive or attenuated influenza virus. As shown in the examples, supplementing influenza vaccines with M2 VLPs induce broad and improved cross protection against multiple influenza A subtypes.

A. VLP vaccines.

Virus-like particles (“VLP”) enhance the immune system, and activate both cellular and humoral immune responses. VLPs closely resemble virions in structure. The non-infectious nature of VLPs and their lack of viral genomic material make them safe for broad and repeated application, particularly for the elderly and infant populations. The self-assembled macrostructure of VLPs presents conformational epitopes to the immune system, which are comparable to those of live virions. The viral glycoproteins are generally unmodified by fixatives and presented in a native conformation, since VLPs are assembled by the normal process of budding on the cell surface. VLPs as particulate antigens are a target for antigen presenting cells such as dendritic cells to capture an antigen and present it to T and B cells. VLPs can be manipulated to incorporate immuno-stimulatory and/or targeting molecules to enhance immune responses.

VLPs closely resemble mature virions, but they do not contain viral genomic material (e.g., viral genomic RNA). Therefore, VLPs are nonreplicative in nature, which make them safe for administration in the form of an immunogenic composition (e.g., vaccine). VLPs can express envelope glycoproteins on the surface of the VLP, which is the most physiological configuration. Since VLPs resemble intact virions and are multivalent particulate structures, VLPs may be more effective in inducing neutralizing antibodies to the envelope glycoprotein than soluble envelope antigens. VLPs can be administered repeatedly to vaccinated hosts, unlike many recombinant vaccine approaches. An example of a VLP vaccine is the baculovirus-derived recombinant human papillomavirus type (HPV-16) L1 VLP, which was manufactured by Novavax, Inc.

VLPs include a viral core/matrix protein that can self-assemble into the VLP core and at least one viral surface envelope glycoprotein expressed on the surface of the VLP. The viral core protein and at least one of the viral surface envelope glycoprotein(s) can be from different viruses. In general, the disclosed VLP includes at least a viral core/matrix protein and at least one viral surface envelope glycoprotein (type 1 or type 2 viral surface envelope glycoproteins). The VLP can include a lipid membrane, viral glycoprotein transmembrane unit, and a matrix protein.

The VLP can further contain at least one adjuvant molecule expressed on the surface of the VLP. The adjuvant molecules can include more than one type of adjuvant molecule.

Chimeric VLPs are VLPs that have at least one viral surface envelope glycoprotein incorporated into the VLP, wherein the viral core protein and at least one viral surface envelope glycoprotein are from different viruses. Chimeric VLPs include VLPs where there are more than one type of viral surface envelope glycoprotein and where one or both are from a different virus than the viral core protein.

Viral proteins include proteins that are capable of self-assembling into the VLP (Freed, E. O., J. Virol., 76, 4679-87, (2002)). In particular, the viral core proteins can include, but are not limited to, a viral Gag protein, in particular, a retrovirus gag protein [e.g. a HIV Gag viral protein (e.g., HIV-1 NL43 Gag (GenBank serial number AAA44987)), a simian immunodeficiency virus (SIV) Gag viral protein (e.g., SIVmac239 Gag (GenBank serial number CAA68379)), or a murine leukemia virus (MuLV) Gag viral protein (e.g., MuLV Gag (GenBank serial number S70394))], a filovirus viral core protein (e.g., an Ebola VP40 viral protein (e.g., Ebola virus VP40 (GenBank serial number AAN37506))), a Rift Valley Fever virus N protein (e.g., RVFV N Protein (GenBank serial number NP049344)), a coronavirus M, E and NP protein (e.g., GenBank serial number NP040838 for NP protein, NP 040835 for M protein, CAC39303 for E protein of Avian Infections Bronchitis Virus and NP828854 for E protein of the SARS virus)), an influenza M1 protein, a paramyxovirus M protein, an arenavirus Z protein (e.g., a Lassa Fever Virus Z protein), and combinations thereof. Appropriate surface glycoproteins and/or viral RNA may be included to form the VLP.

VLPs can be made by introducing into a cell a viral surface glycoprotein expression vector, a viral core protein expression vector, and optionally an adjuvant molecule expression vector, and allowing for expression of the viral surface envelope surface glycoprotein and the optional adjuvant molecule, whereby the VLP is formed by the cells.

In general, the viral protein sequence and the corresponding polynucleotide sequence can be found in GenBank and the access numbers can be obtained online at National Center for Biotechnology Information (NCBI). Variants that are substantially homologous to these viral proteins and viral proteins having conservative substitutions of the above referenced viral proteins can also be incorporated into VLPs to enhance the immunogenic characteristics of the VLPs.

The viral surface envelope glycoprotein, or at least at portion of the viral surface envelope glycoprotein, is disposed (e.g., expressed) on the surface of the VLP so that it can interact with target molecules or cells to produce immunogenic responses (e.g., antibody production).

The disclosed VLPs contain M2 protein, or an immunogenic fragment thereof containing at least the extracellular domain of M2 (M2e), or an immunogenic conservative variant thereof. In general, the viral surface envelope glycoprotein sequence and the corresponding polynucleotide sequence can be found in GenBank and the access numbers can be obtained online at NCBI. Immunogenic variants that are substantially homologous to the viral surface envelope glycoproteins and viral surface envelope glycoproteins having conservative substitutions of these viral surface envelope glycoproteins can also be incorporated into VLPs to enhance the immunogenic characteristics of VLPs. An immunogenic variant of a reference protein is one that would provoke an immune response to the reference protein. Therefore,

Preferably, the VLP contains M1 protein, i.e., is an M1 VLP. In other embodiments, the viral core contains heterologous matrix/core protein, e.g., lentiviral Gag protein. In some embodiments, VLPs are prepared containing one or more influenza hemaglutinin (HA) proteins and/or one or more influenza neuraminidase (NA) proteins. However, in preferred embodiments, the disclosed VLPs do not contain HA or NA. In some embodiments, the M2 protein is the sole influenza antigen on the surface of the disclosed VLPs.

Also disclosed is a virosome containing M2 protein integrated within a lipid membrane. Other particles or constructs that can present M2 peptide on the surface of the particle or construct are further considered for use in the disclosed compositions and methods.

B. Influenza Matrix Protein 2 (M2)

Influenza M2 protein is a small transmembrane protein present on the surfaces of influenza virions. It possesses an ion channel activity which plays a role in the process of viral entry into cells. It is also the only component present on the surfaces of influenza A virus particles that is highly conserved among virus strains, and has therefore been of interest as a possible vaccine antigen to induce broad-spectrum immunity to prevent influenza virus infection. Previous efforts to develop a vaccine to elicit effective immune responses to M2 have not been very successful, in part because of the small size of the protein, its presence in the virus in low copy numbers, and probably because it is masked by the other viral surface proteins (HA and NA) which are larger and more abundant.

M2 of influenza A viruses is a tetrameric type III membrane protein, exhibiting pH-dependent proton transport activity. It is expressed at high density in the plasma membrane of infected cells and is accessible to M2e-specific antibodies, but only a few copies are incorporated into the envelope of viruses. The small M2e epitope is highly conserved, but is poorly immunogenic. Studies have shown that although M2e-specific antibodies were not able to prevent infection, they restricted subsequent virus replication and reduced illness and death. Some improved designs protected immunized mice from lower lethal dose virus challenges (De Filette et al., 2006a; De Filette et al., 2008a; De Filette et al., 2008b; De Filette et al., 2006b; Eliasson et al., 2008; Fan et al., 2004; Heinen et al., 2002; Liu, Li, and Chen, 2003; Neirynck et al., 1999).

The wild type M2 protein contains three structural domains: an amino-terminal extracellular domain (M2e, comprising 24 residues), a transmembrane domain (TM, 19 residues), and a cytoplasmic domain (CT, 54 residues). The M2 protein in the disclosed VLPs preferably contains at least the amino-terminal extracellular domain (M2e). In some embodiments, the M2 proteins in the VLPs are full-length protein. In other embodiments, the M2 proteins in the VLPs are fragments containing at least amino acids 1-24 of M2. The M2 protein can in some embodiments have one or more conservative amino acid substitutions within amino acids 1-24 of M2.

1. Fusion Proteins

In some embodiments, the VLPs incorporate an M2 fusion protein. The M2 fusion protein in the VLP can contain full-length M2 protein, or an immunogenic fragment thereof (e.g., M2e). The M2 fusion protein can contain a tetramerization domain, a membrane anchor domain, a signal peptide, an adjuvant protein, or any combination thereof.

The fusion protein in the VLP can have the following formula:

X₁-(M2e)ₙ-X₂-X₃,

wherein X₁ is optionally an adjuvant protein, a signal peptide, or a combination thereof;

wherein “M2e is an M2 extracellular domains;

wherein “n” is an integer equal to or greater than one (e.g., 1, 2, 3, 4, 5, 6, 7, 8, 9, 10);

wherein “X₂” is optionally an oligomerization (e.g., dimerization, tetramerization) domain;

wherein “X₃” is optionally an adjuvant protein, a membrane anchor domain (e.g., transmembrane domain), or a combination thereof; and

wherein “-” is a peptide bond or amino acid linker.

2. Multimeric M2e Protein

In some embodiments, the M2 fusion protein is multimeric. For example, in some embodiments, the M2 fusion protein contains two, three, four, or more M2 subunits (e.g., M2e). For example, the M2 protein can be dimeric or tetrameric. In some embodiments, the fusion protein contains M2 protein (e.g., M2e) from two or more different influenza virus subtypes (i.e., is heterogeneous). In some embodiments the multiple subunits of M2 are contiguous (i.e., linked in tandem). In other embodiments the multiple subunits of M2 are separated by one or more amino acid linkers and/or peptide domains.

For example, a wild-type M2e fragment having the sequence MSLLTEVETPIRNEWGCRCNDSSDP (SEQ ID NO:1) was modified to delete the initiating methionine and to replace the cysteines with serine to create the following modified M2e sequence: SLLTEVETPIRNEWGSRSNDSSDP (SEQ ID NO:2). Therefore, the disclosed VLPs can include a fusion protein having two or more M2e peptides having the sequence SEQ ID NO:2, or a conservative variant thereof. For example, the M2e peptides can be joined by peptide bond or amino acid linker(s).

Four of the modified M2e sequences were then linked together via flexible linkers to create the following tetrameric M2e (4.M2e) fusion protein:

(SEQ ID NO: 3)
VDHMCAAASLLTEVETPIRNEWGSRSNDSSDPAAGTSAAASLLTEVETPI
RNEWGSRSNDSSDPAAALQAAASLLTEVETPIRNEWGSRSNDSSDPAAA
ACAAASLLTEVETPIRNEWGSRSNDSSDPAAAACKL.

The M2e sequences in SEQ ID NO:3 are underlined, and the other sequences are the flexible linkers. Therefore, the disclosed VLP can include a fusion protein having the amino acid sequence SEQ ID NO:3.

Other matrix proteins such as VSV M, paramyxovirus M, HIV Gag, MMTV core protein, or lassa virus Z core protein can also be used.

3. Membrane Anchors

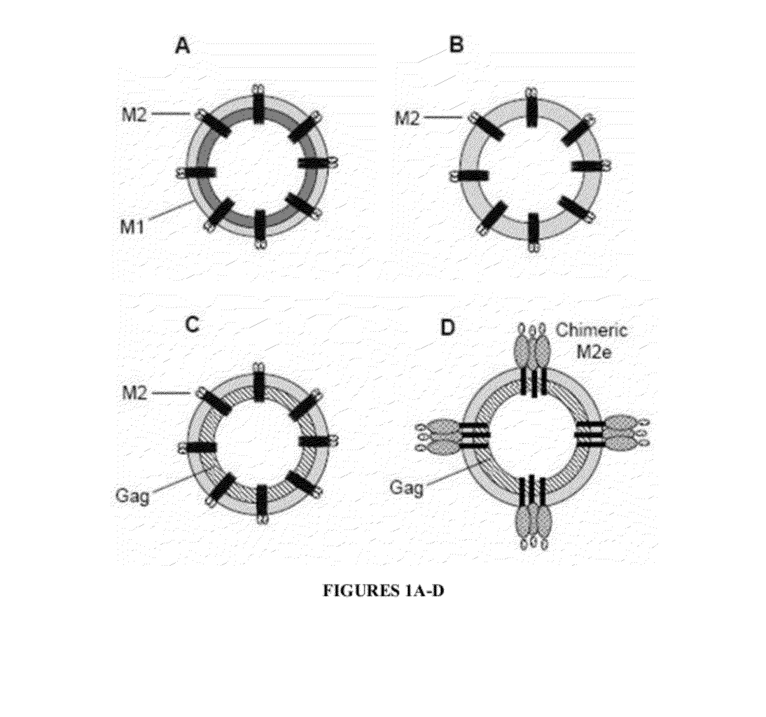

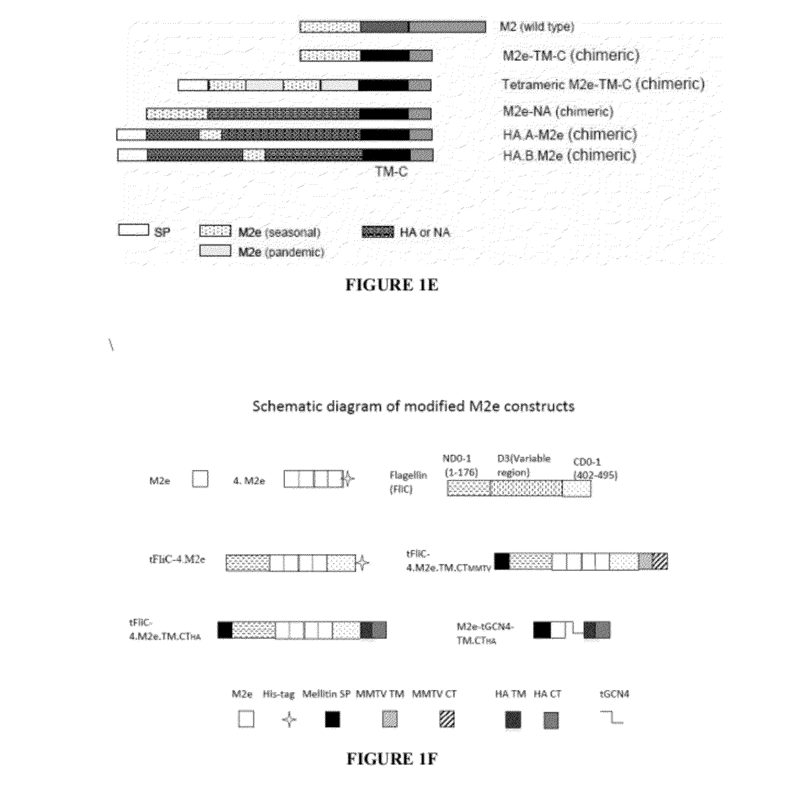

The M2 fusion protein preferably contains a membrane anchor domain to immobilize the M2 peptide (e.g., M2e) on the surface of the VLP. For example, the membrane anchor can be a transmembrane domain (and optionally the cytoplasmic domain) of a viral envelope protein. In some embodiments, the fusion protein contains the transmembrane domain of M2, or a fragment thereof sufficient to anchor the fusion protein to the VLP membrane. In other embodiments, the transmembrane domain derives from HA or NA. Therefore, in some embodiments, the fusion protein contains a fragment of HA, such as the transmembrane domain and optionally the cytoplasmic domain of HA. In some embodiments, the fusion protein contains a fragment of NA, such as the transmembrane domain and optionally the cytoplasmic domain of NA. In preferred embodiments, the M2 protein is linked to the N-terminus of the HA or NA protein fragments. In some embodiments, the M2 protein is incorporated within the antigenic site of the HA or NA proteins. Exemplary constructs are described in FIG. 1F.

Incorporation of wild type M2 or tandem repeats of M2 ectodomain into VLPs can be enhanced, for example, by replacing transmembrane and/or cytoplasmic domains of wild type M2 or tandem repeats of M2 ectodomain of M2 with sequences of other proteins known to be localized in cholesterol-rich lipid raft domains on cell surfaces, such as those from influenza hemagglutinin. Examples of suitable sequence include the HIV Vpu* transmembrane domain (*Vpu transmembrane domain has the following sequence:

| | |
|---|---|
| MQPIQIAIVALVVAIIIAIVVWSIVII. | (SEQ ID NO: 6) |

The wild type M2 or tandem repeats of M2 ectodomains to the HA2 domain of influenza hemagglutinin can include transmembrane and cytoplasmic domains, including highly conserved fusion peptides. The transmembrane and cytoplasmic domains of wild type M2 or tandem repeats of M2 ectodomain of M2 can be replaced with those from MMTV Env protein.

4. Tetramerization Domain

By engineering a tetramerization structure and a membrane-anchor, M2e can be presented in a tetrameric and membrane-bound form, mimicking the structure and microenvironment of native M2 protein. In a preferred embodiment, the tetramerization domain is engineered into M2e, to produce VLPs containing modified M2e chimeric protein mimicking the tetrameric structure of native M2 protein. For example, the M2e fusion protein can contain a modified leucine zipper tetramerization motif of GCN4 (tGCN4).

5. Adjuvants

In some embodiments, the immunogenicity of the disclosed VLP is enhanced by incorporation of an adjuvant. The adjuvant can be incorporated into the composition containing the VLP, into the VLP (e.g., by expressing a membrane-anchored form of an adjuvant protein), or as a domain in an M2 fusion protein.

In preferred embodiments, the adjuvant is flagellin, the natural ligand of the toll-like receptor 5 (TLR5). For example, the flagellin can be a modified membrane-anchored form.

Adjuvants are used to increase the immune response to an antigen and can boost vaccine efficacy to address or overcome the decrease in effectiveness when a heterovariant strain is in circulation. MF59-adjuvanted influenza vaccine is an adjuvanted influenza vaccine available for use in humans in some parts of the world, but not in the U.S. Only one adjuvant is currently approved by FDA for use in humans: alum, a mixture of aluminum salts. It was found that alum effectively adjuvanted M2 VLPs, when intranasally administered, resulting in a significant adjuvant effect on enhancing the cross protection (FIG. 9). Adjuvants that are effective, yet do not elicit too vigorous reactions either systemically or at the site of injection, are essential for efficacy. Therefore, in some embodiments, the composition containing the disclosed VLP further contains an adjuvant such as Alum.

Toll-like receptors (TLRs) are an important family of innate immune receptors that recognize pathogen-associated molecular patterns (PAMPs) that are required for microbial fitness. They are the best characterized type of pattern recognition receptor (PPR) expressed on antigen-presenting cells (APCs). Following the PAMP-PRR interaction, an intracellular signaling cascade is initiated, leading to the production of type-1 interferon (IFN) and proinflammatory cytokines, which mediate the innate antiviral immune responses. Innate immunity provides an immediate reply to a viral challenge, which also controlling the later antigen specific-adaptive immune response.

Flagellin is the primary protein component of the highly complex flagellar structures that extend from the outer membrane of Gram-negative organisms. It has been well documented that TLR5 recognizes a conserved site on flagellin. TLR5 is detected in epithelia in the lung and gut and is also highly expressed in residual dendritic cells (DCs) such as those in the lamina propria of the intestine. DCs are critical for induction of an effective adaptive immune response, connecting the innate and adaptive immune system by presenting antigens from the site of exposure to naïve lymphocytes in secondary locations. It has been proven that flagellin promotes the maturation of DCs as well as their migration to lymph nodes. Acting as the natural agonist of TLR5, flagellin is a highly potent and effective adjuvant in humans and non-human primates. It is a strong inducer of innate immune effectors such as cytokines and nitric oxide. When administrated by a mucosal route, flagellin stimulated innate immunity in the mouse lung. Furthermore, flagellin-induced activation of adaptive immune responses is known to influence the presentation of antigens and the activation of a cellular immune response.

The protein nature of flagellin is an advantage for many immuno-therapeutic applications due to its ease of manipulation. Flagellin augmentation of antibody responses, as well as T cell responses, have been demonstrated for purified natural, recombinant, and truncated forms. When a modified flagellin was fused to the West Nile Virus envelope protein, this recombinant protein vaccine candidate elicited stronger specific antibodies, and protected mice from lethal live virus challenge.

6. Exemplary M2e Constructs

Exemplary M2e constructs expressing fusion proteins with the domain structures shown in FIGS. 1A-E were generated and are shown in FIG. 1F.

The tFliC-4.M2e construct contains the Flagellin (FliC) ND0-1 domain at its N-terminus followed by the 4.M2e sequence (SEQ ID NO:3) with the CD0-1 domain of FliC at the C-terminus. The construct also contains a C-terminal His-tag for use in purification of the fusion protein.

The tFliC-4.M2e.TM.CT_{MMTV} construct contains an N-terminal mellitin signal peptide followed by the FliC ND0-1 domain, followed by the 4.M2e sequence (SEQ ID NO:3), followed by the CD0-1 domain of FliC, followed by the transmembrane domain of MMTV, with the cytoplasmic tail of MMTV at the C-terminus.

The tFliC-4.M2e.TM.CT_{HA} construct contains an N-terminal mellitin signal peptide followed by the FliC ND0-1 domain, followed by the 4.M2e sequence (SEQ ID NO:3), followed by the CD0-1 domain of FliC, followed by the transmembrane domain of HA, with the cytoplasmic tail of HA at the C-terminus.

The M2e-tGCN4-TM.CT_{HA} construct contains an N-terminal mellitin signal peptide followed by a single M2e or tandem repeats 4.M2e, followed by tGCN4 tetramerization domain (ELKQIEDKLEEILSKLYHIENELARIKKLLGE, SEQ ID NO:4), followed by the transmembrane domain of HA, with the cytoplasmic tail of HA at the C-terminus.

One exemplary fusion protein has a mellitin signal peptide, a single copy M2e or tandem repeats 4.M2e, a modified leucine zipper tetramerization motif of GCN4 (tGCN4) and the influenza HA transmembrane/cytoplasmic domains (see Example 1). The resulting tetrameric M2e was incorporated into M1-derived VLPs. A membrane-anchored, variable region-truncated flagellin was also constructed and co-incorporated into these VLPs as an adjuvant.

7. Preparing Fusion Proteins

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. Thus, any of the disclosed functional domains can be used to design a fusion protein.

Fusion proteins can be created through the joining of two or more genes which originally coded for separate proteins. Translation of this fusion gene results in a single polypeptide with function properties derived from each of the original proteins. Recombinant fusion proteins can be created artificially by recombinant DNA technology. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either. If the two entities are proteins, often linker peptides are also added which make it more likely that the proteins fold independently and behave as expected.

Alternatively, internal ribosome entry sites (IRES) elements can be used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

C. Viral Vaccines

In some embodiments, the disclosed VLPs improve cross protection against multiple subtypes of influenza A virus subtypes and/or strains when co-administered with traditional viral vaccine. The inactivated or live, attenuated human influenza A virus preferably contains an H1N1 strain, an H3N2 strain, or a combination thereof.

Two types of seasonal influenza vaccine are licensed by the Food and Drug Administration (FDA) for use in the United States: trivalent influenza vaccine (TIV) and live, attenuated influenza vaccine (LAIV). In some embodiments, the inactivated influenza A virus is a TIV. Non-limiting examples of TIVs include AGRIFLU (Novartis), FLUZONE (Sanofi Pasteur), AFLURIA (CS Limited), FLULAVAL (ID Biomedical Corporation of Quebec), FLUARIX (GlaxoSmithKline), and FLUVIRIN (Novartis). In some embodiments, the attenuated influenza A virus a LAIV. For example, the LAIV can be FLUMIST (MedImmune Vaccines).

Both seasonal LAIV and TIV generally contain three strains of influenza viruses that are antigenically equivalent to the annually recommended strains: one influenza A (H3N2) virus, one influenza A (H1N1) virus, and one influenza B virus. Each year, before influenza season starts, one or more virus strains in the vaccine might be changed on the basis of global surveillance for influenza viruses and the emergence and spread of new strains.

TIV is generally injected into the muscle of the upper arm or thigh. It can be used for healthy people who are 6 months of age or older, and it can be used in people with chronic medical conditions and women who are pregnant. Healthy people are defined as people who do not have an underlying medical condition that predisposes them to influenza complications. LAIV is generally given as a nasal spray. It can be used for healthy people 2-49 years of age who are not pregnant.

D. Pharmaceutical Compositions

The disclosed compositions, including VLPs, can be used therapeutically in combination with a pharmaceutically acceptable carrier. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Pharmaceutical compositions may include carriers, thickeners, diluents, buffers, preservatives, and surface active agents in addition to the VLPs. Pharmaceutical compositions may also include additional ingredients such as adjuvants (e.g., alum). Preparations for injection include sterile aqueous or non-aqueous solutions, suspensions, and emulsions. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Preservatives and other additives may also be present such as, for example, antimicrobials, anti-oxidants, and chelating agents.

### III. Methods of Making the Antigenic Formulations

The disclosed VLPs can be produced using mammalian, plant, yeast, insect or bacterial expression systems, such as, but not limited to, a baculovirus expression system (Yamshchikov et al., (1995) Virology: 214, 50-58) or recombinant poxvirus expression system (MVA) (Wyatt et al., (1996), Vaccine: 15, 1451-1458), using any recombinant vector including viral, plasmid and bacterial vectors such as recombinant VSV, recombinant adenovirus, and recombinant DNA expression vectors.

In general, VLPs can be produced by simultaneously introducing into a cell a viral matrix/core protein expression vector, a viral surface envelope glycoprotein expression vector, and optionally an adjuvant molecule expression vector. The expressed viral core protein self-assembles into a VLP that incorporates the viral surface envelope glycoprotein and optionally the adjuvant molecule. The viral surface envelope glycoprotein and optionally the adjuvant molecule are expressed and disposed on the VLP surface. Thereafter, the cell produces the VLP. The cells may be selected from, but are not limited to, insect cells (e.g., Spodoptera frugiperda Sf 9 and Sf21 cells), and mammalian cells (e.g., EL4 cells and HeLa cells). The expression elements for expressing the viral core protein, viral surface envelope glycoprotein, and adjuvant molecule can also be included together in a single expression vector, or can be included in two or more expression vectors.

In some embodiments, the VLP of the disclosed method is produced by coinfecting cells (e.g., insect cells) with one or more recombinant vectors (e.g., baculoviruses) expressing the M1 and M2 proteins (e.g., full-length or M2e fusion proteins), culturing the insect cells under physiological conditions, and purifying VLPs from cell culture supernatants.

In general, the viral protein expression vector can be produced by operably linking a coding sequence for a viral protein of a virus to an appropriate promoter (e.g., an early promoter, late promoter, or hybrid late/very late promoter). The viral protein expression vector can also be modified to form a viral protein expression construct. In addition, the viral surface envelope glycoprotein expression vector can be produced by operably linking a coding sequence for a viral surface envelope glycoprotein of a virus to an appropriate promoter (e.g., early promoter, late promoter, or hybrid late/very late promoter). The viral surface envelope glycoprotein expression vector can be modified to form a viral surface envelope glycoprotein expression construct. Similarly, the adjuvant molecule expression vector can be produced by operably linking a coding sequence for an adjuvant molecule to an appropriate promoter (e.g., early promoter, late promoter, or hybrid late/very late promoter). The adjuvant molecule expression vector can be modified to form an adjuvant molecule expression construct.

In other embodiments, polynucleotide sequences encoding for a viral core protein (e.g., M1), at least one viral surface envelope glycoprotein (e.g., M2 or M2e fusion protein), and optionally at least one adjuvant molecule (e.g., FliC) can be included in a single expression vector, or in two or more expression vectors. The one or more expression vectors can be introduced into a host cell, the proteins can be expressed in the cell, whereby the cell forms the VLP. In some embodiments, each of the polynucleotide sequences encoding for the viral core protein, the viral surface envelope glycoprotein, and optionally the adjuvant molecule is operably linked to an appropriate promoter (e.g., a baculovirus promoter, a recombinant Modified Vaccinia Ankara (MVA) promoter, a CMV promoter, an EF promoter, an adenovirus promoter, a recombinant VSV promoter, a recombinant adenovirus promoter, a recombinant alphavirus promoter, or a recombinant DNA expression vector). Appropriate promoters include, but are not limited to, a constitutive or inducible promoter; an early, late, or hybrid late/very late promoter.

The following studies to test immunogenicity used a gene encoding the membrane-anchored wild type M2 or tandem 4 repeats of M2e (4.M2e) generated by fusing encoding sequences for a mellitin signal peptide, a modified leucine zipper tetramerization motif (tGCN4) and the influenza HA transmembrane/cytoplasmic domains in frame. The resulting tandem 4 repeats or a single copy of M2 was incorporated into M1-derived VLPs. A membrane-anchored, variable region-truncated flagellin was also constructed and co-incorporated into these VLPs as an adjuvant. Naïve M2-containing VLPs and a recombinant histidine-tagged tetrameric M2e protein were also generated for comparison. M2e VLPs and wild type M2 VLPs were tested for eliciting strong humoral and mucosal responses by intranasal or systemic inoculation in a mouse model, and immunized mice were challenged with a panel of H1N1 and H3N2 viruses, and a H5N1 virus. A general method is to link M2e to any protein adjuvant in an y position: N-terminus, C-terminus or internal.

### IV. Methods of Administering Vaccine

The vaccine may be administered by traditional vaccine routes, such as by intramuscular, intradermal, or subcutaneous injection, by microneedles, or by intranasal administration. The vaccine may be given as a single dose, or as a priming dose followed by booster(s), as is standard in the industry for immunization with influenza vaccine.

The exact amount of the compositions required will vary from subject to subject, depending on the species, age, weight and general condition of the subject, the particular VLP used, and its mode of administration. An appropriate amount can be determined by one of ordinary skill in the art using only routine experimentation given the teachings herein. Effective dosages and schedules for administering the compositions may be determined empirically, and making such determinations is within the skill in the art. The dosage ranges for the administration of the compositions are those large enough to induce an immune response. The dosage should not be so large as to cause adverse side effects, such as unwanted cross-reactions or anaphylactic reactions.

It is expected that 2 to 50 μM2 protein incorporated into VLPs would be effective in inducing broadly protective immunity in humans. An anticipated dosage is 10 to 50 μg of M2 in VLPs per dose, based on the use of approximately 10-20 μg of total proteins of M2 VLPs in a mouse. 10-20 μg of total proteins of M2 VLPs are estimated to contain 1-2 μg of M2 in VLPs, administered to a mouse weighing between 16 to 20 gram.

### EXAMPLES

### Example 1

### Generation of Modified M2e Constructs

4.M2e

Fusion proteins containing four M2e sequences linked in tandem and separated by flexible linkers as shown in FIGS. 1A-1E were generated using standard molecular biology techniques. The M2e sequence: MSLLTEVETPIRNEWGCRCNDSSDP (SEQ ID NO:1) was modified to delete the initiating methionine and to replace the cysteines with serine to create the following modified M2e sequence: SLLTEVETPIRNEWGSRSNDSSDP (SEQ ID NO:2).

Four of the modified M2e sequences were then linked together via flexible linkers to create the following fusion protein:

(SEQ ID NO: 3)
VDHMCAAASLLTEVETPIRNEWGSRSNDSSDPAAGTSAAASLLTEVETPI
RNEWGSRSNDSSDPAAALQAAASLLTEVETPIRNEWGSRSNDSSDPAAA
ACAAASLLTEVETPIRNEWGSRSNDSSDPAAAACKL.

The M2e sequences in SEQ ID NO:3 are underlined, and the other sequences are the flexible linkers.

Additional M2e Constructs

Additional M2e constructs expressing fusion proteins with the domain structures shown in FIGS. 1A-E were also generated, as shown in FIG. 1F. The tFliC-4.M2e construct contains the Flagellin (FliC) ND0-1 domain at its N-terminus followed by the 4.M2e sequence (SEQ ID NO:3) with the CD0-1 domain of FliC at the C-terminus. The construct also contains a C-terminal His-tag for use in purification of the fusion protein. The tFliC-4.M2e.TM.CT_{MMTV} construct contains an N-terminal mellitin signal peptide followed by the FliC ND0-1 domain, followed by the 4.M2e sequence (SEQ ID NO:3), followed by the CD0-1 domain of FliC, followed by the transmembrane domain of MMTV, with the cytoplasmic tail of MMTV at the C-terminus. The tFliC-4.M2e.TM.CT_{HA} construct contains an N-terminal mellitin signal peptide followed by the FliC ND0-1 domain, followed by the 4.M2e sequence (SEQ ID NO:3), followed by the CD0-1 domain of FliC, followed by the transmembrane domain of HA, with the cytoplasmic tail of HA at the C-terminus. The M2e-tGCN4-TM.CT_{HA} construct contains an N-terminal mellitin signal peptide followed by a single M2e or tandem repeats 4.M2e, followed by tGCN4 tetramerization domain (ELKQIEDKLEEILSKLYHIENELARIKKLLGE, SEQ ID NO:4), followed by the transmembrane domain of HA, with the cytoplasmic tail of HA at the C-terminus.

### Example 2

### M2 protein based Influenza VLPs Vaccine

It was hypothesized that incorporation of an engineered tetrameric M2e into virus-like particles (VLPs) lacking HA and NA, would yield highly immunogenic VLPs. Their immunogenicity would be further enhanced by incorporation of an adjuvant such as a modified membrane-anchored form of flagellin, the natural ligand of the toll-like receptor 5 (TLR5). It was predicted that the resulting VLPs would elicit high titers of M2-specific antibodies and thereby confer protection against infection by a range of influenza A viruses.

Methods and Materials

A gene encoding the membrane-anchored a single M2e or tandem repeat 4.M2e was generated by fusing encoding sequences for a mellitin signal peptide, a single copy M2e or tandem repeats 4.M2e, a modified leucine zipper tetramerization motif of GCN4 (tGCN4) and the influenza HA transmembrane/cytoplasmic domains in frame, as described in Example 1. The resulting tetrameric M2e was incorporated into M1-derived VLPs. A membrane-anchored, variable region-truncated flagellin was also constructed and co-incorporated into these VLPs as an adjuvant. Naïve M2-containing VLPs and a recombinant histidine-tagged tetrameric M2e protein were generated for comparison.

Influenza virus-like particles (VLPs) were produced in Sf9 insect cells by co-expressing the matrix protein M1 and the surface glycoproteins hemagglutinin (HA) and neuraminidase (NA) using the recombinant baculovirus expression system. VLPs are produced in various combinations of influenza components including the M2. Examples of VLPs include M2 only, M1+M2, M1+M2+HA, M1+M2+HA+NA.

Expression and Purification of M2E Fusion Proteins and Generation of Virus-Like Particles (VLPs).

Histidine-tagged 4.M2e and flagellin-4.M2e proteins were purified from the E. coli protein expression system. To construct the his-tagged 4.M2e and flagellin-4.M2e, the full-length encoding genes described above were cloned into expression vector pET-22b(+) (Novigen) under T7 promoter and lac operator with NdeI/Xho1 in frame with histidine tag. The validation of the resulted construct was confirmed by DNA sequencing. To produce soluble protein, an E. coli strain BL was used for the production of proteins by 1 mM IPTG induction. Five hours post-induction, cells were harvested and used for the preparation of protein with Nickel-bead column (Qiagen). Affinity purification of His-tagged proteins was performed by following the manufacturer's instruction.

His-tagged soluble tetrameric M2e was produced by a recombinant baculovirus (rBV) protein expression system. DNA fragments encoding Mellitin signal peptide, M2e and tGCN4 tetrameric domain were fused by overlapping PCR to generate the full-length gene encoding the His-tagged soluble tetrameric M2e. The resulting gene was cloned into pFastBac-1 (invitrogen) and used for making rBV with the bac-to-bac insect cell protein expression kit (Invitrogen). His-tagged soluble tetrameric M2e was produced in Sf9 insect cell culture infected with rBV, and concentrated from the cell culture supernatant using Nickel-bead column.

VLPs were produced in Sf9 insect cell culture by co-infection of rBVs expressing 4.M2e-tFliC-TM.CT_{MMTV or HA} and M1(4.M2e-tFliC-TM.CT_{MMTV}/M1 VLPs), tFliC_{HA} and M1(tFliC/M1 VLPs), respectively.

### Example 3

### M2e fusion Proteins and VLPs Generate IgG and IgA Antibodies in Immunized Mice

Materials and Methods

As shown in Table 1, mice were immunized with 10 μg of 4.M2e protein, 10 μg of 4.M2e-tFliC fusion protein, 50 μg of 4.M2e-tFliC-TM.CT_{MMTV}/M1 virus-like particles (VLPs), or a mixture of 10 μg of 4.M2e protein with 10 μg of tFliC/M1. Mice were immunized either intramuscularly (IM), intranasally (IN) or by microneedle (MN). Six mice were immunized per group, 3 times each at 4 week intervals. Serum samples were collected after each immunization.

TABLE 1
M2e Immunizations

| Groups | Antigen Forms | Doses (μg) | Route |
|---|---|---|---|
| 4.M2e | Protein | 10 | IM |
| 4.M2e | Protein | 10 | IN |
| 4.M2e-tFliC | Fusion Protein | 10 | IM |
| 4.M2e-tFliC | Fusion Protein | 10 | IN |
| 4.M2e-tFliC | Fusion Protein | 10 | MN |
| 4.M2e-tFliC-MMTV/ | VLPs | 50 | IM |
| M1 | | | |
| 4.M2e-tFliC-MMTV/ | VLPs | 50 | IN |
| M1 | | | |
| 4.M2e + tFliC/M1 | Mixture | 10 + 10 | IM |
| 4.M2e + tFliC/M1 | Mixture | 10 + 10 | IN |

IM, intramuscular;
IN, intranasal;
MN, microneedle.
Three immunizations were performed in 4 week intervals.
Serum samples were collected two weeks after each immunization.

Serum IgG endpoint titers were determined by ELISA using assay plates coated with 100 μl/well of recombinant tandem M2e (5 μg/μl). Immune serum samples were 2×stepwise diluted started at 100×. Diluted samples were applied to plates for antibody-binding. Bound antibodies were detected by binding horseradish peroxidase (HRP)-goat anti-mouse IgG antibody and color was developed with tetramethylbenzidine (TMB) substrate. The greatest dilution which presented an OD450 2× higher than that of the negative control (naïve group) was designated as the endpoint titer.

Serum IgG endpoint titers were determined by cell surface ELISA using MDCK cells infected with PR8 at an MOI of 1. The MDCK cells were washed with PBS and fixed with 10% formalin at 12 hours post-infection. Bound antibodies were detected by binding HRP-goat anti-mouse IgG antibody and color was developed with TMB substrate.

Lungs from immunized mice were collected and extracted with 1 ml PBS containing 0.05% Tween 20 per lung twice. Lung extract IgA endpoint titers were determined by ELISA using assay plates coated with 100 μl/well of recombinant tandem M2e (5 μg/μl). Immune serum samples were 2×stepwise diluted started at 100×. Diluted samples were applied to plates for antibody-binding. Bound antibodies were detected by HRP-goat anti-mouse IgA antibody and color was developed with TMB substrate. The greatest dilution which presented an OD450 2× higher than that of the negative control (naïve group) was designated as the endpoint titer.

Results

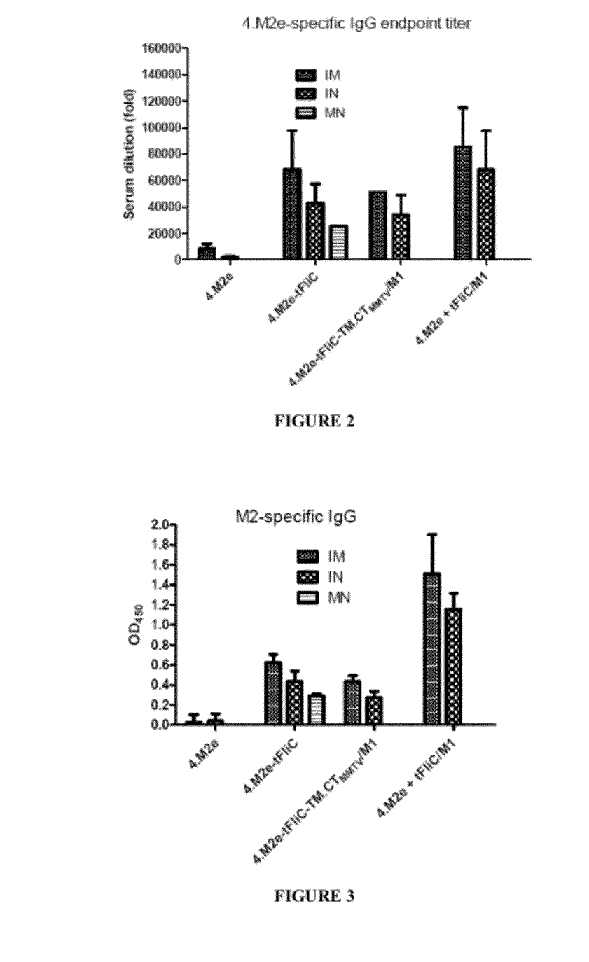

FIG. 2 is a bar graph showing serum 4.M2e-specific IgG endpoint titers determined by ELISA from mice immunized with 10 μg of 4.M2e protein, 10 μg of 4.M2e-tFliC fusion protein, 50 μg of 4.M2e-tFliC-TM.CT_{MMTV}/M1 virus-like particles (VLPs), or a mixture of 10 μg of 4.M2e protein with 10 μg of tFliC/M1, for mice immunized either intramuscularly (IM), intranasally (IN) or by microneedle (MN), as indicated.

FIG. 3 is a bar graph showing serum M2-specific IgG endpoint titers determined using transfected MDCK cells from mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1, immunized either intramuscularly (IM), intranasally (IN) or by microneedle (MN), as indicated.

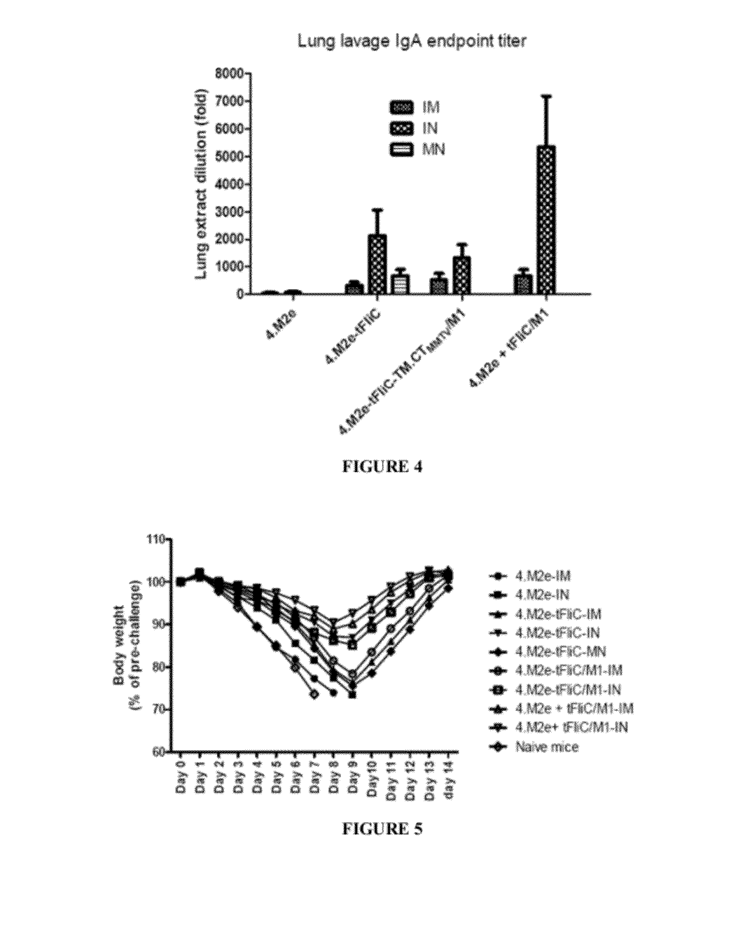

FIG. 4 is a bar graph showing lung lavage IgA endpoint titers from mice immunized with 10 μg of 4. M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1, for mice immunized either intramuscularly (IM), intranasally (IN) or by microneedle (MN), as indicated.

In conclusion, fusion protein containing tandem repeats 4.M2e fused with flagellin TLR5 binding domains distributed in both termini of the tandem (sandwich fusion, 4.M2e-tFliC) is highly immunogenic compared to tandem repeats (4.M2e), inducing high level of systemic antibody responses by IN, IM or MN immunization and enhanced mucosal antibody responses by IN immunization. Membrane-anchored form of the fusion protein (4.M2e-tFliC-TM.CT_{MMTV}) can be incorporated into influenza M1-derived VLPs. Resulting VLPs induced high levels of immune responses compared to the tandem repeats 4.M2e by IN or IM immunization.

### Example 4

### Testing of M2e VLPS with Viral Challenge

M2e fusions proteins and M2e VLPs were tested for eliciting strong humoral and mucosal responses by intranasal, systemic or microneedle inoculation in a mouse model, and immunized mice were challenged with PR8 (H1N1) and Philippines (H3N2) viruses to evaluate the broad protective efficacy of the vaccine candidates.

Materials and Methods

Naive mice or mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1, were subsequently infected with 5×LD₅₀ A/PR8. Mice were immunized either intramuscularly (IM), intranasally (IN), or by microneedle (MN).

Results

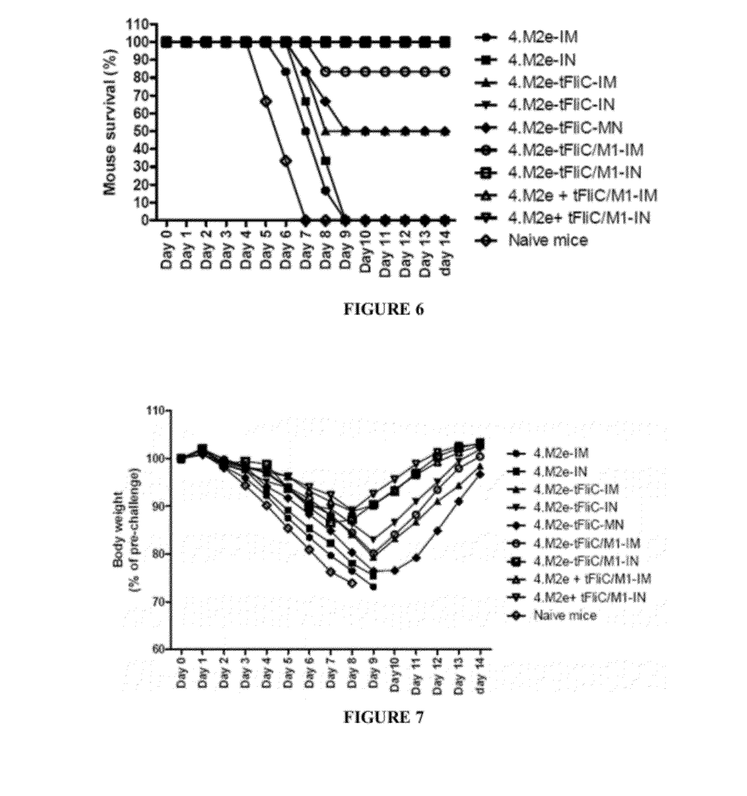

FIG. 5 is a line graph showing change in body weight (% of pre-challenge) as a function of time (days). FIG. 6 is a line graph showing survival of mice (%) as a function of time (days) in naive mice or mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1, and subsequently infected with 5×LD₅₀ A/PR8.

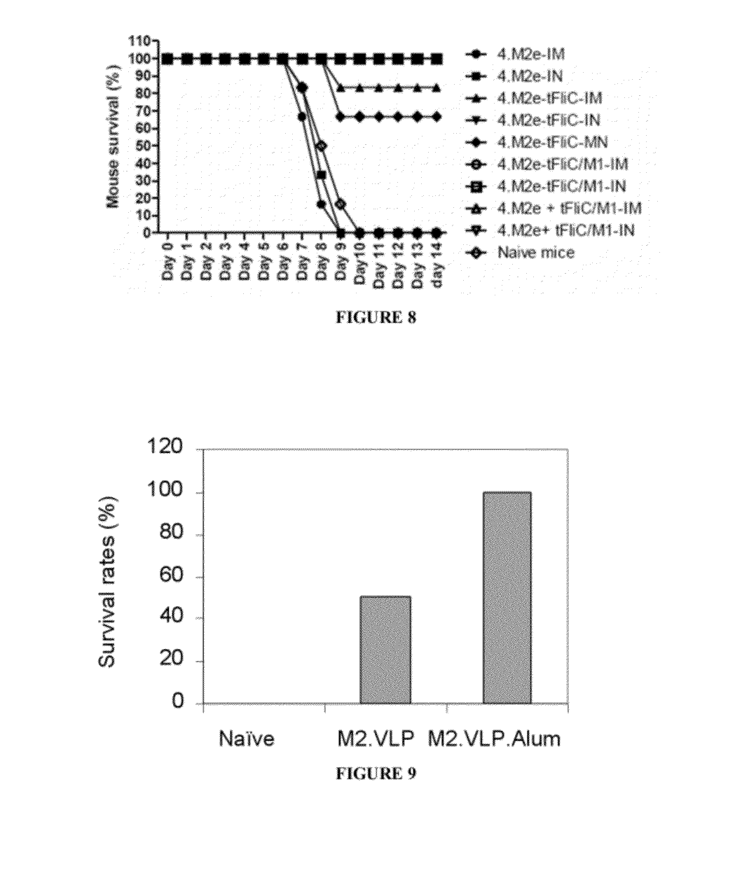

FIG. 7 is a line graph showing change in body weight (% of pre-challenge) as a function of time (days). FIG. 8 is a line graph showing survival of mice (%) as a function of time (days) in naive mice or mice immunized with 10 μg of 4.M2e protein, 10 μg of 4.M2e-tFliC fusion protein, 50 μg of 4.M2e-tFliC-TM.CT_{MMTV}/M1 VLPs, or a mixture of 10 μg of 4.M2e protein with 10 μg of tFliC/M1, and subsequently infected with 5×LD₅₀ A/Philippines.

In conclusion, M2e fused with the TLR5 ligand flagellin, or M2e adjuvanted by flagellin VLPs, or M2e fusion protein incorporated into M1-derived VLPs induced broad immunity when IM, IN or MN administered, conferring subtypic cross-protection in the mouse model. Immunized mice showed decreased morbidity and mortality when challenged by lethal doses of PR8 (H1N1) or Philippines (H3N2) viruses, demonstrating the universal protective efficacy of M2e fusion or modified M2e VLPs.

### Example 5

### M2 VLP Vaccine can be Adjuvant with Alum to Enhance Cross Protection

Materials and Methods

The M2-encoding gene was cloned into a BV shuttle vector (pFastbac) and a rBV expressing the wild type M2 was generated. Influenza VLPs containing M2 were produced in insect cells coinfected with rBVs expressing M1 and M2, were purified using sucrose gradient ultracentrifugation, and characterized by western blot using anti-M2 monoclonal antibody 14C2. Next, we tested the adjuvant effect of Alum on inducing heterosubtypic cross protection by influenza VLPs containing the M2 protein of A/WSN strain (M2 VLPs). Groups of mice (n=6, BALB/c mice) were intranasally immunized with 10 μg of M2 VLPs alone or co-immunized with 10 μg of M2 VLPs and 50 μg of Alum at weeks 0 and 4. Four weeks post boost vaccination, groups of mice were challenged with a lethal dose of A/Philippines virus (6×LD₅₀). Body weight changes and survival rates were determined.

Results

FIG. 9 is a bar graph showing the survival rates. The adjuvant effect of Alum on inducing antibodies specific to M2 peptide antigen was significant after intranasally co-immunization of mice. The Alum-adjuvanted group of mice immunized intranasally with M2 VLPs were 100% protected against a lethal dose of the heterosubtypic strain A/Philippines (H3N2). In contrast, the group of mice intranasally immunized with M2 VLPs in the absence of Alum showed only 50% protection against the heterosubtypic A/philippines/82 strain. Also, the Alum-adjuvanted group showed less sign of illness (approximately 15 to 18% body weight loss) than a non-adjuvanted group (approximately 22 to 25% body weight loss). All naïve (unimmunized) mouse controls (n=6) died around days 6 to 7 post infection.

M2 VLPs provide protection against lethal infection with heterosubtypic strains. Alum adjuvant showed improved cross protection indicating its role as a mucosal adjuvant.

### Example 6

### M2 VLPs can be Superior to Influenza Virus in Inducing M2-Specific Immune Responses

Materials and Methods

Although M2 is suggested to be a promising target for developing a universal vaccine, immune responses specific to M2 are very weak or not easily detected after influenza vaccination probably due to the immuno-dominant effects of HA. To investigate the role of M2 in inducing cross protection, we produced influenza VLPs containing M2 (derived from A/WSN). To determine the immunogenicity of influenza VLPs containing M2, groups of mice (6 BALB/c mice per group) were intranasally immunized with M1-derived VLPs containing M2 (M2 VLP) or PR8 HA (H1 VLP). For comparison, additional groups of mice were immunized intranasally with egg-grown inactivated A/PR8 virus alone or supplemented with M2 VLPs. All groups of mice were immunized with 10 μg (total protein) of an indicated vaccine at week 0 and 4. At week 12 and 28 post boost immunization, M2-specific IgG antibodies were determined by ELISA using M2 ectodomain peptide as a coating antigen

Results

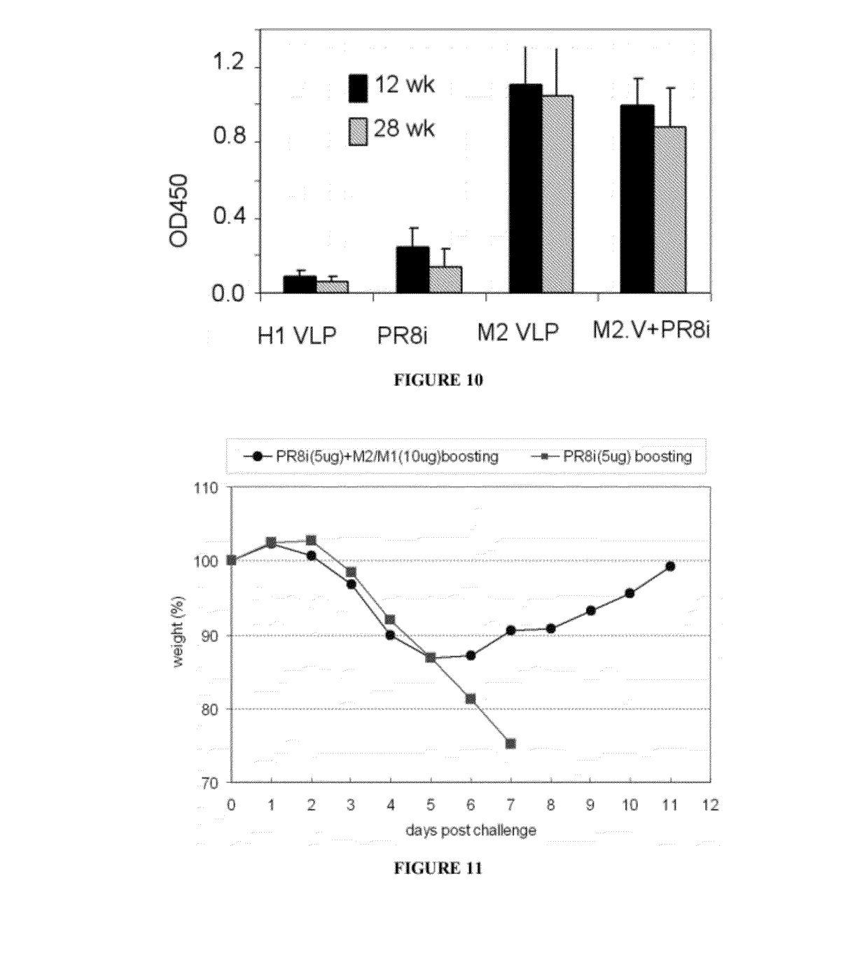

FIG. 10 is a bar graph showing M2-specific antibody responses. The H1 VLP group without M2 did not show M2 specific antibody responses. Most importantly, levels of M2-specific antibodies were significantly higher in M2 VLP-immunized mice than those in inactivated virus immunized mice and were maintained for over 7 months after immunization. Importantly, the M2 VLP supplemented inactivated A/PR8 virus group induced high levels of antibodies specific to M2.

These results suggest that VLPs containing M2 can be more effective in inducing immune responses to M2 than the inactivated whole virus and are likely to induce long-lived immune responses to M2. Also, these results provide a rationale that supplementing M2 VLPs to the vaccine might confer enhanced cross protection.

### Example 7

### M2 VLP Supplemented Vaccine Significantly Improved the Cross Protective Vaccine Efficacy

Materials and Methods

The current inactivated influenza vaccine is not effective in providing protection against antigenically drifted or shifted strains. It was therefore tested whether the addition of M2 VLPs to the inactivated influenza vaccine as a supplement would enhance the cross protection against heterosubtypic virus challenge. Groups of mice (n=6, BALB/c mice) were intranasally immunized with inactivated influenza H1N1 virus vaccine (A/PR8) or a mixture of inactivated influenza H1N1 virus (A/PR8) plus M2 VLPs (M2 derived from H1N1 virus A/WSN) at weeks 0 and 4. Five weeks post boost immunization, vaccinated mice were challenged with a lethal dose of the H3N2 heterosubtypic strain A/Philippines or the 2009 pandemic virus.

Results

FIG. 11 is a line graph showing the effects of M2 VLPs on enhancing the cross protection against A/Philippines/82 virus. The group of mice that was immunized intranasally with inactivated A/PR8 virus was not protected and all mice died by day 7 post challenge. Importantly, all A/PR8 immunized mice supplemented with M2 VLPs were 100% protected against the lethal infection with A/Philippines H3N2 virus (12 LD₅₀) although they showed moderate body weight loss of approximately 12%. We observed significantly higher levels of serum antibodies specific to M2 peptide antigen in the group of M2 VLP supplemented mice than those in mice with inactivated vaccine only.

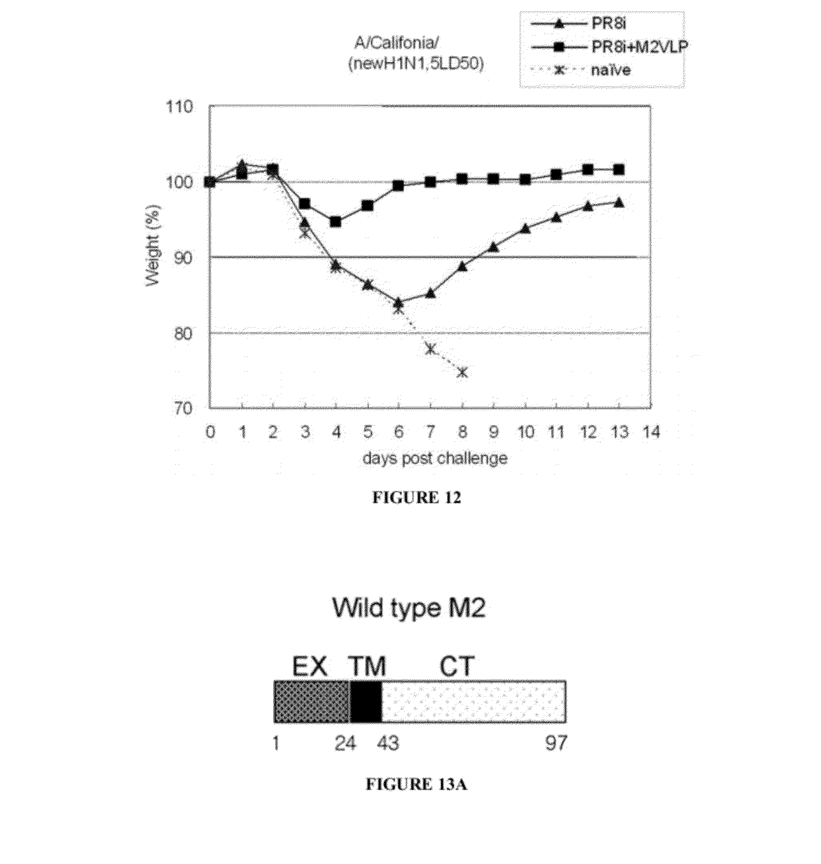

FIG. 12 is a line graph showing the effects of M2 VLPs on enhancing the cross protection against the 2009 pandemic A/California/2009 virus. The all naïve mice died of infection by day 8. Seasonal vaccines are not expected to provide protection against the 2009 new H1N1 pandemic virus. The group of mice immunized with inactivated A/PR8 virus alone exhibited a severe loss up to 18% in body weight. However, the M2 VLP supplemented vaccine group showed a significantly improved protection against the 2009 new pandemic virus resulting in a loss of less than 5% in body weight.

Addition of influenza M2 VLPs significantly improved the efficacy of vaccines in inducing cross protection. Induction of improved heterosubtypic protection by supplementing the vaccine with M2 VLPs has significant implication for developing more effective influenza vaccines inducing broadly cross protective immunity.

### Example 8

### M2 VLP Supplemented Vaccine Significantly Improved the Cross Protective Vaccine Efficacy

Materials and Methods

Viruses and VLPs

Influenza A viruses A/PR/8/34 (H1N1), A/Philippine/2/82 (H3N2), A/California/04/09 (H1N1), A/WSN/33 (H1N1), and reassortant A/Vietnam/1203/04 (H5N1; HA and neuraminidase were derived from A/Vietnam/1203/04, and the remaining backbone genes from A/PR/8/34 virus) were propagated in chicken eggs and used for challenge studies.

Preparation and Characterization of M2 VLPs

M2 VLPs were produced as previously described (Kang S M, et al. (2009) PLoS ONE 4:e4667; Song J M, et al. (2010) Virology 405:165-175). Briefly, an rBV expressing the WT M2 gene derived from influenza A/WSN/33 (H1N1) virus was generated and grown in sf9 insect cells (CRL-1711; American Type Culture Collection). M2 VLPs were produced in insect cells coinfected with rBVs expressing influenza M1 and M2 proteins. VLPs were purified from culture supernatants by using Quixstand hollow fiber-based ultrafiltration (GE Healthcare) and sucrose gradient ultracentrifugation. Similarly, HIV VLPs were produced in insect cells coinfected with rBVs expressing HIV Env and Gag proteins (Wang B Z, et al. (2007) J Virol 81:10869-10878). Negative staining of influenza A/PR/8/34 virus and M2 VLP for EM images was carried out as described (Quan F S, et al. (2007) J Virol 81:3514-3524).

Animal Experiments

For animal experiments, 6- to 8-wk-old female BALB/c mice (n=9; Harlan Laboratories) were intranasally immunized with 2 μg total protein of A/PR8-inactivated virus alone (PR8i) or inactivated A/PR8 vaccine (2 μg) supplemented with M2 VLPs or HIV VLPs at weeks 0 and 4. Blood samples were collected at 3 wk after each immunization. To investigate heterosubtypic protective immunity, immunized mice were challenged with a lethal dose (6×LD₅₀) of A/California/04/09, A/Phil, or reassortant H5N1 A/Vietnam/1203/04 influenza viruses at 4 wk after boost immunization. Mice were euthanized if their body weight loss exceeded 25%.

Determination of Antibody Responses, Lung Viral Titers, and INF-Secreting Cells

Serum antibody responses were determined by ELISA using synthetic M2e peptide (2 μg/mL), recombinant H5 HA protein (4 μg/mL), or whole inactivated virus (4 μg/mL) as a coating antigen as previously described (Song J M, et al. (2010) Virology 405:165-175). The M2 extracellular domain (M2e) peptide (17 aa; 2-18; SLLTEVETPIRNEWGCR; SEQ ID NO:5) was synthesized at the Emory University Biochemical Core Facility (Atlanta, Ga.). M1-specific antibody responses were determined using a peptide pool (2 μg/mL) for the M1 protein derived from influenza A/New York/348/2003 (H1N1) virus (cat. no. NR-2613; BEI Resources). Antibody titers were expressed as the averages of the highest dilution factors that give twofold higher SD compared with those in naive sera. For determination of HAI titers, serum samples from individual mice (n=6) were collected at 4 wk after boost immunization. HAI titers of immune sera against homologous (H1N1 A/PR8), heterologous (H1N1 A/WSN), and heterosubtypic viruses (H5N1 A/Vietnam, H3N2 A/Phil) were determined by standard methods using 4 HA units of virus as described previously (Quan F S, et al. (2010) J Virol 84:7760-7769). Lung viral titers were determined using a plaque assay in Madin-Darby canine kidney cells as previously described (Quan F S, et al. (2007) J Virol 81:3514-3524). The limit of virus detection was 50 pfu (1.69 Log10) per milliliter of organ homogenate. IFN-γ-secreting cell spots were determined on Multiscreen 96-well plates (Millipore) coated with cytokine-specific capture antibodies as described (Quan F S, et al. (2007) J Virol 81:3514-3524). Briefly, 10⁶ spleen cells per well were cultured with or without M2e peptide (10 μg/mL) as an antigenic stimulator. After 36 h culture, the number of IFN-γ-secreting cells was counted using an ImmunoSpot reader (Cellular Technology).

Virus Neutralization Assays

Virus-neutralizing activities of immune sera were determined by a conventional plaque reduction assay as described (Quan F S, et al. (2007) J Virol 81:3514-3524). For virus growth inhibition assay, approximately 80% confluent Madin-Darby canine kidney cells were infected at a multiplicity of infection of 0.1 of influenza A/Philippines/82 virus (H3N2). The virus solution was washed out and replaced with DMEM containing immune sera of various dilutions. Culture media were harvested at day 2 after infection, and the viral yield was estimated by hemagglutination activity assay using chicken red blood cells as described (Song J M, et al. (2010) Virology 405:165-175).

Statistical Analysis

To determine statistical significance, two-tailed Student t test and one-way ANOVA were used when comparing two or more different groups, respectively. P value less than 0.05 was considered to be significant.

Results

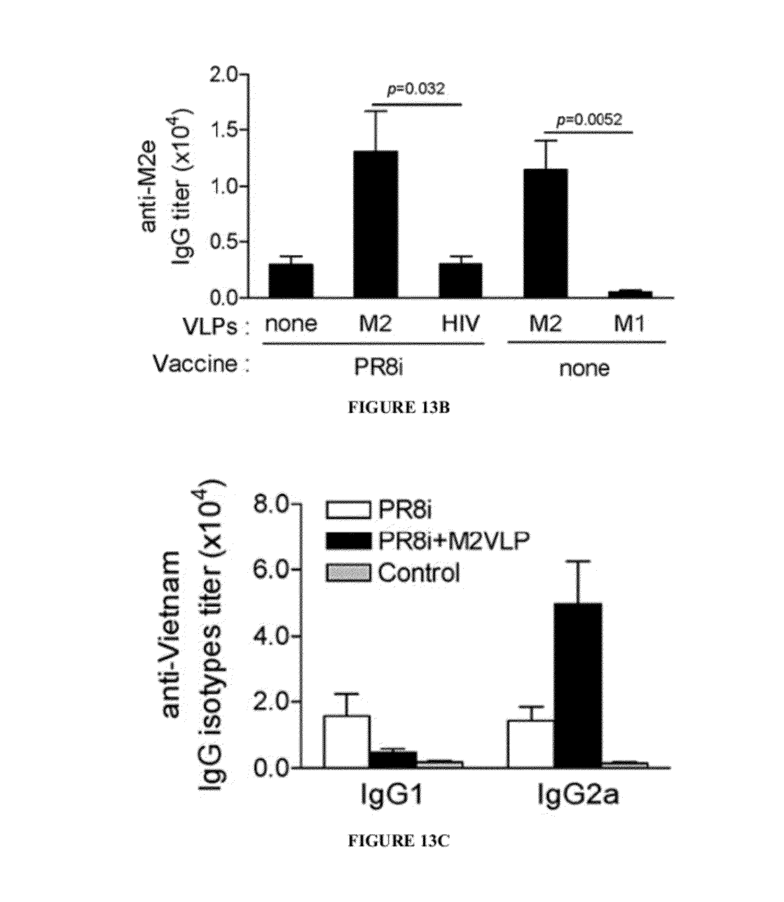

VLPs containing M2 from A/WSN/33 (M2 VLPs) virus were produced in insect cells co-infected with recombinant baculoviruses (rBVs) expressing the WT M1 and M2 proteins (FIG. 13A). M2 VLPs showed similar size as influenza virions as examined by EM. The M2 content in VLPs was found to be comparable to that of virus. Here, these VLPs were investigated as a supplement for influenza vaccines to enhance the immune responses to M2 and increase the breadth of protection against influenza viruses.

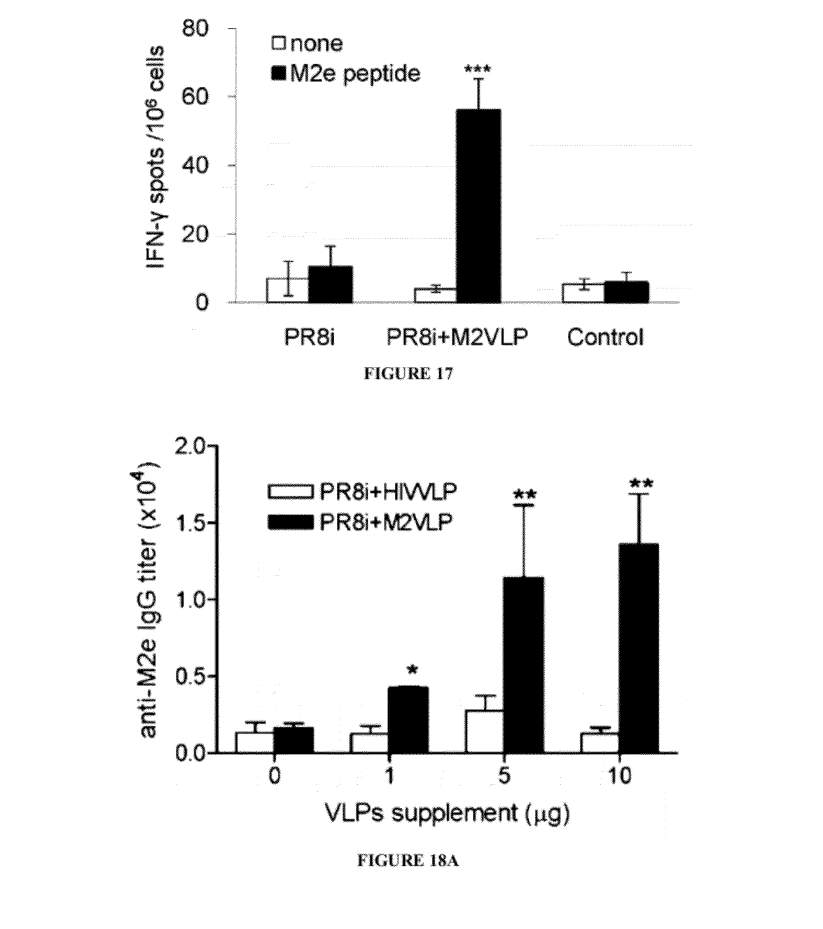

To determine the effects of M2 VLPs on inducing cross-reactive responses, groups of mice were intranasally immunized with 2 μg of inactivated A/PR/8/34 virus (PR8i) alone or inactivated A/PR8 virus (μg) supplemented with 10 μg of M2 VLPs (PR8i+M2VLP). M2-specific antibodies (FIG. 13B) and INF-γ-secreting cell responses (FIG. 17) were induced in the M2 VLP-supplemented group at levels that were significantly higher than those in the PR8i-alone group (FIG. 13B). As controls, vaccination with HIV VLPs as supplement did not increase M2-specific immune responses above the low levels seen in the PR8i group and M1 VLPs without M2 did not induce M2-specific antibody responses, indicating that the increased immune responses are the result of M2 VLPs (FIG. 13B). Interestingly, vaccination with PR8i+M2VLP induced similar levels of M2e-specific antibodies as M2 VLPs alone (FIG. 13B). These results show that supplementing an inactivated influenza vaccine with M2 VLPs as a conserved antigenic target can significantly improve the immune response to M2.

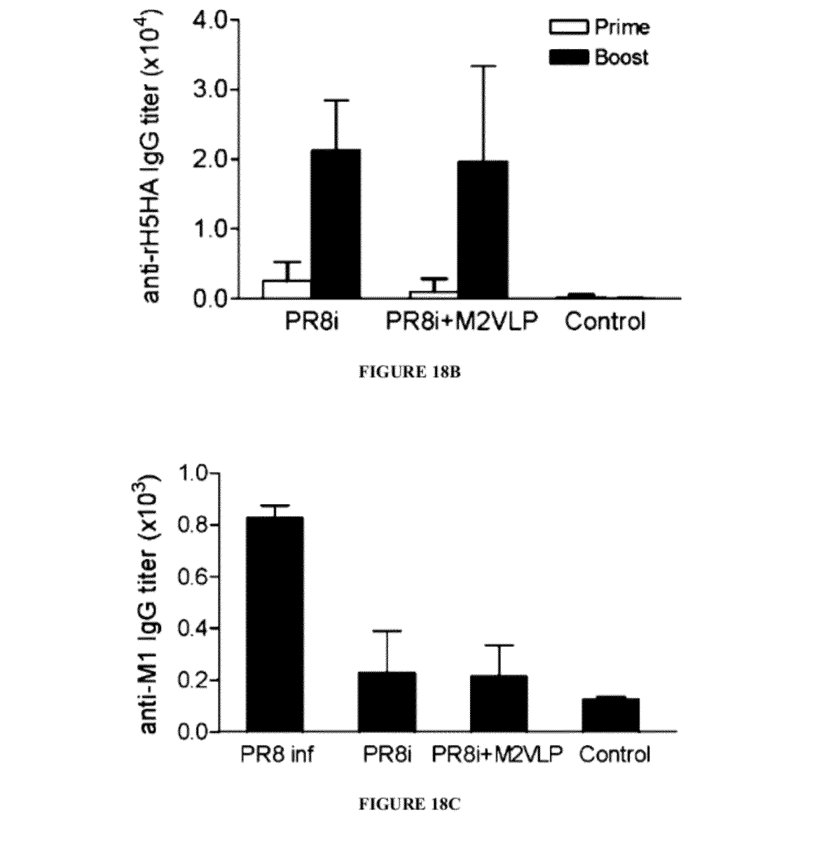

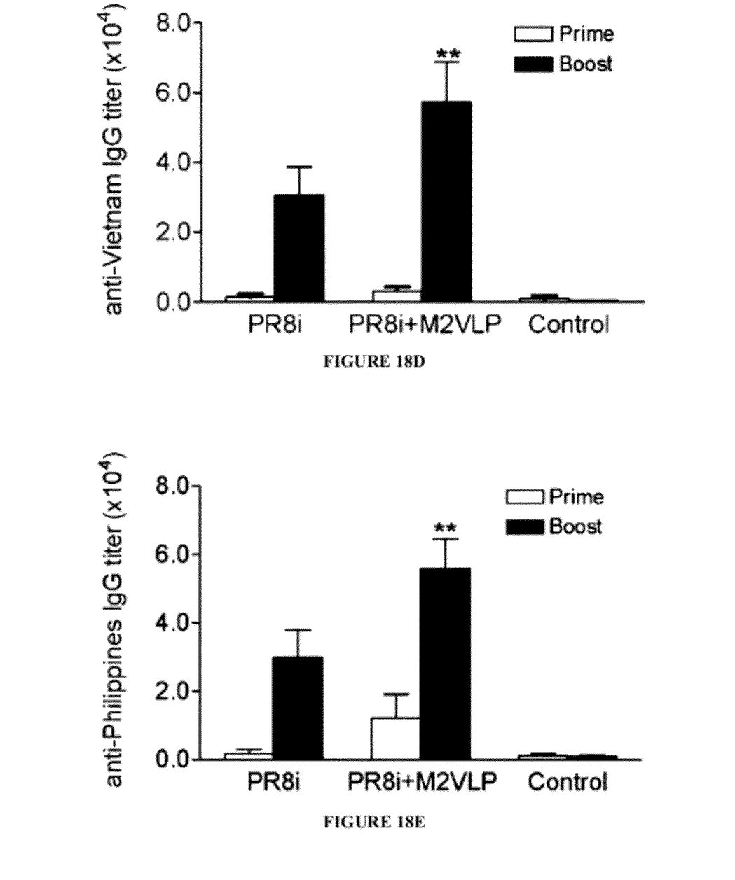

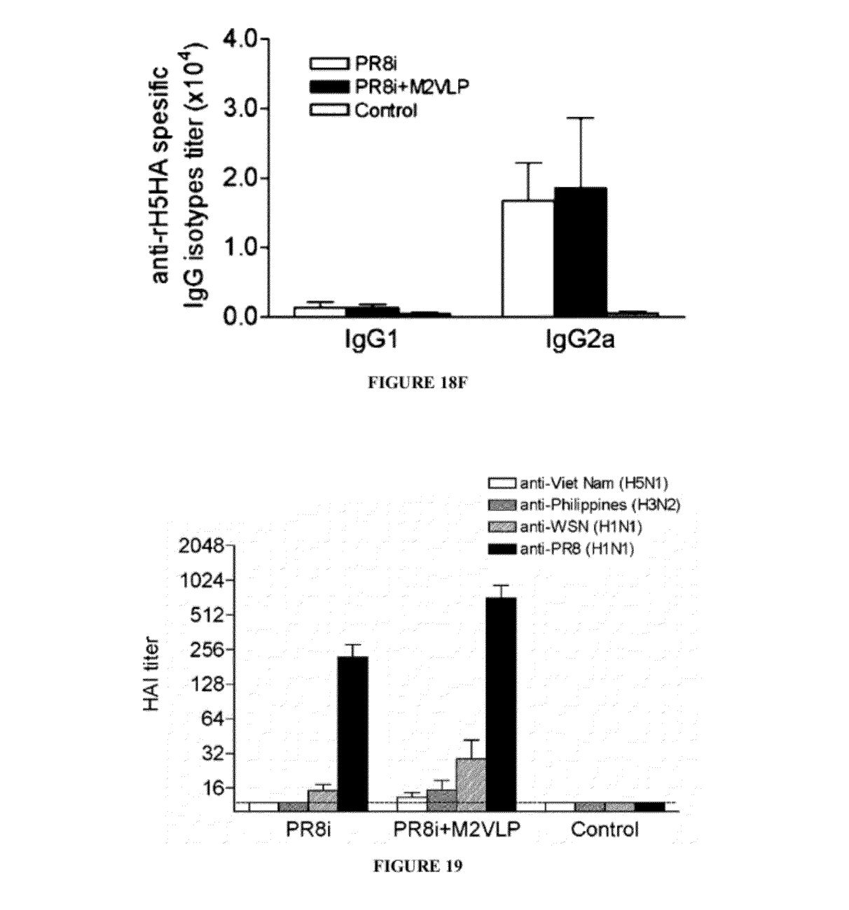

Dosage effects of VLP supplements (1, 5, 10 μg) indicated that lower dose of M2 VLPs, but not unrelated HIV VLPs, could be used as supplemental vaccine to enhance immune responses to M2 (FIG. 18A). No significant differences were observed in binding antibody levels to the purified H5 HA protein (FIG. 18B) or M1 peptide pool (FIG. 18C) between the PR8i+M2VLP and PR8i groups. Importantly, it was noted that immune sera from M2 VLP supplemented vaccination (PR8i+M2VLP) enhanced the breadth of cross-reactivity to different subtypes of H3N2 and H5N1 viruses (FIGS. 18D and 18E). Therefore, immunization with the M2 VLP supplemented vaccine significantly increases the cross-reactive response to heterosubtypic influenza A viruses.

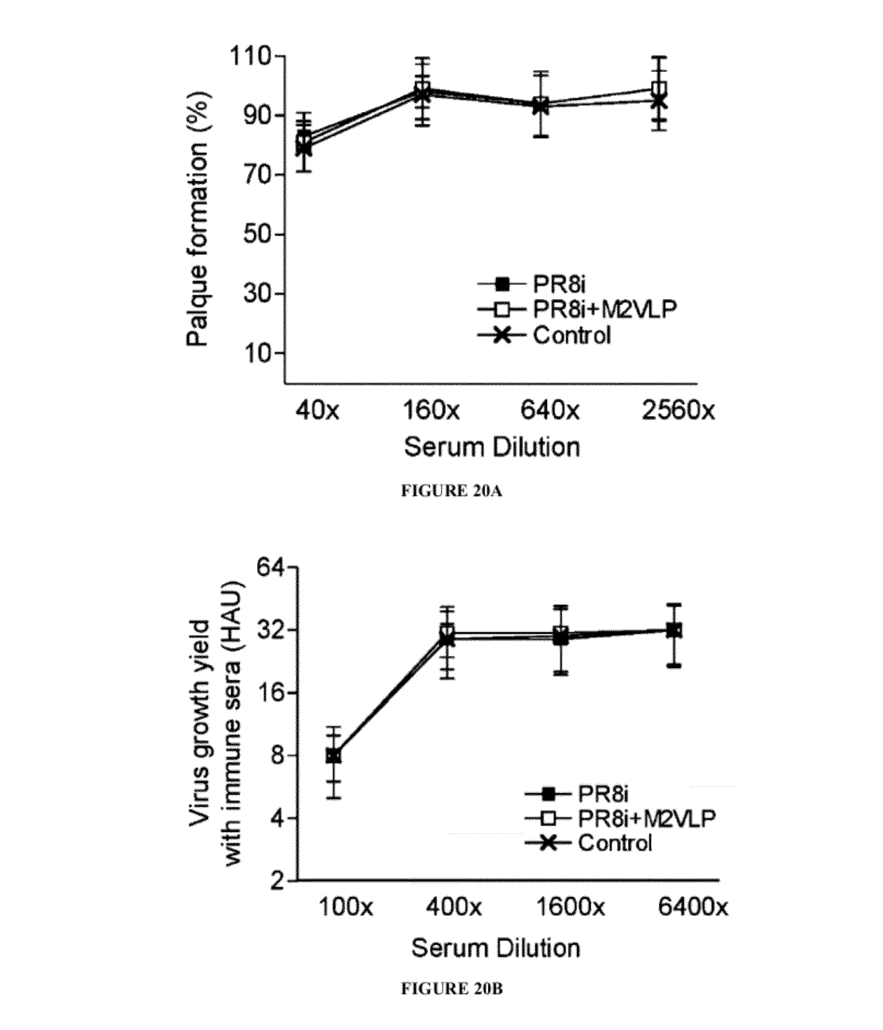

The PR8i+M2 VLP-supplemented group also showed higher levels of IgG2a versus IgG1 specific to the H5N1 A/Vietnam/1203/04 virus (ratio of approximately 10) compared with the PR8i group (ratio of approximately 1; FIG. 13C), but no difference in binding antibody levels to the H5 HA protein (FIGS. 18B and 18F). The supplementation of inactivated A/PR8 vaccine with M2 VLPs did not induce significant cross-reactive hemagglutination inhibition (HAI) activities against the heterosubtypic H3N2 A/Philippines/82 (A/Phil) or H5N1 A/Vietnam/1203/04 viruses (FIG. 19). Also, immune sera from PR8i+M2VLP or PR8i groups did not show virus neutralizing activity or growth inhibition effects against the H3N2 heterosubtypic virus at significant levels (FIG. 20). Therefore, enhanced binding antibodies to M2e did not correlate with cross-reactive HAI response or heterosubtypic virus neutralizing activity. These results indicate that M2 VLPs can be an effective supplement for influenza vaccines to improve the breadth of cross reactivity to antigenically different viruses by enhancing M2 immunity, and that IgG2a isotype antibody recognizing M2e was preferentially induced by the M2 VLP supplement.

### Example 9

### M2 VLP Supplementation Improves Heterologous and Heterosubtypic Protection

The potential effects of M2 VLP supplements on inducing cross-protection was further investigated.

Materials and Methods

At 8 wk after boost, naive or immunized mice were challenged with a lethal dose (6×LD₅₀) of heterosubtypic H3N2 A/Phil virus. Lung viral titers at day 4 after challenge with A/Phil were determined. To further analyze the breadth of cross protection, protection against a lethal dose of reassortant H5N1 A/Vietnam/1203/04 virus (a “reassortant virus” is a virion containing deoxyribonucleic acid from one virus species and a protein coat from another) or the 2009 H1N1 A/California virus was tested.

Results

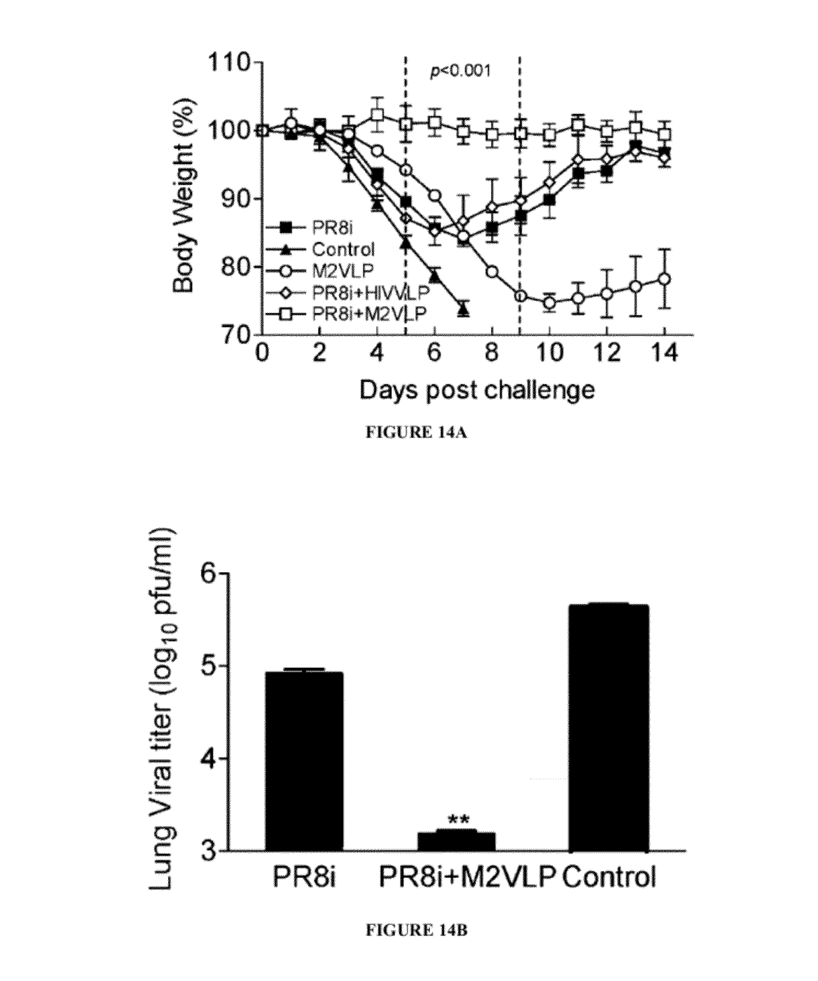

All mice in the control group died from lethal infection (FIG. 14A). Mice immunized with inactivated A/PR8 virus or M2 VLPs alone showed severe loss of body weight of as much as 16%, although 100% of mice survived infection (FIG. 14A). The HIV VLP-supplemented PR8i vaccine group exhibited significant weight loss, indicating lack of effective protection against the heterosubtypic virus (FIG. 14A). In contrast, mice that were supplemented with M2 VLPs (PR8i+M2VLP) did not show signs of disease or weight loss.

To better appreciate the heterosubtypic protective efficacy observed by the addition of M2 VLPs, lung viral titers at day 4 after challenge with A/Phil were determined (FIG. 14B). The PR8i+M2VLP group had significantly lower lung viral titers by more than 200-fold compared with the control group and by 52-fold compared with the PR8i group.

To further analyze the breadth of cross protection, protection against a lethal dose of reassortant H5N1 A/Vietnam/1203/04 virus or the 2009 H1N1 A/California virus was tested. The PR8i vaccinated group showed significant loss in weight of as much as 11%, whereas the PR8i+M2VLP group did not show any loss in body weight after challenge with the H5N1

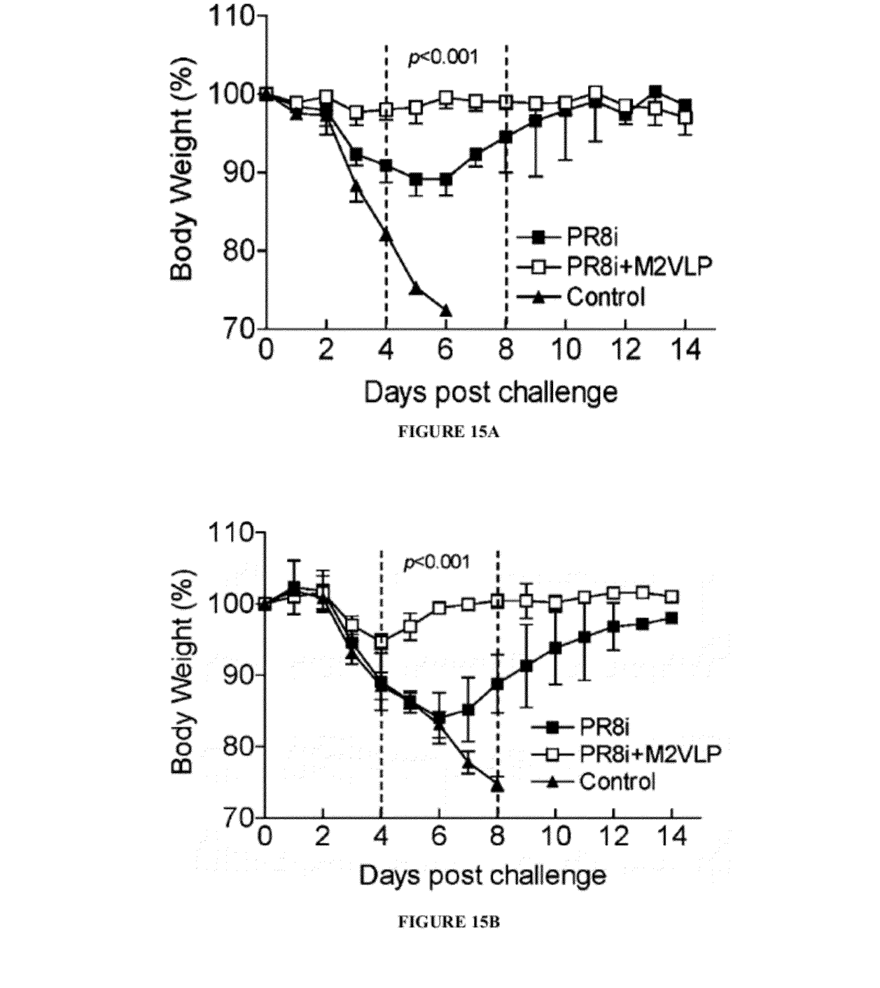

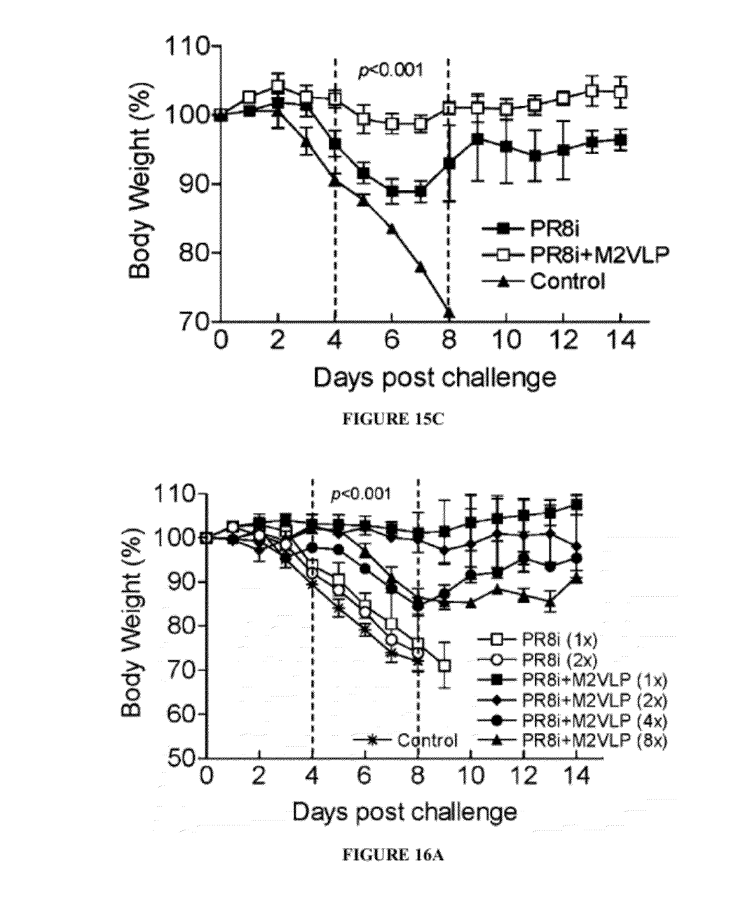

In the case of challenge with the 2009 pandemic H1N1 A/California/2009 virus (FIG. 15B), the mice immunized with inactivated A/PR8 alone were severely ill, as evidenced by greater than 17% body weight loss. The M2 VLP supplemented group showed only slight transient loss in body weight. These results indicate that supplementing an inactivated vaccine with M2 VLPs significantly improves the cross-protective efficacy of the vaccine against heterologous and heterosubtypic influenza A viruses.

### Example 10

### M2 VLP Supplementation Induces Long-Lasting Cross-Protective Immunity

Materials and Methods

To determine the duration of cross protection, mice immunized with A/PR8 alone or A/PR8 plus M2 VLPs were challenged with lethal dose of heterosubtypic A/Philippines virus (6×LD₅₀) at 7 mo after vaccination.

Results

All mice in the control group suffered severe body weight loss and died after challenge (FIG. 15C). The mice immunized with inactivated A/PR8 virus vaccine showed more than 12% loss in body weight and significant delay in body weight recovery. In contrast, mice in the M2 VLP-supplemented group did not show weight loss and all survived the challenge infection. These results indicate the induction of long-lasting cross-protective immunity by influenza vaccine supplemented with M2 VLPs.

### Example 11

### Clodronate Treatment Abrogates Cross Protection by M2 VLP Immune Sera

Materials and Methods

Clodronate Treatment and Cross-Protective Efficacy of Immune Sera

Naive mice were infected with a mixture of a lethal dose of influenza virus and pooled sera collected from vaccinated mice as described previously (Quan F S, et al. (2008) J Virol 82:1350-1359; Quan F S, et al. (2010) PLoS ONE 5:e9161). Briefly, 25 μL of A/Phil virus (6×LD₅₀) mixed with 25 μL immune sera with or without heat inactivation (56° C., 30 min) were used to infect naive mice (n=4, BALB/c), and body weight changes and survival rates were monitored daily.

Liposome-encapsulated clodronate was prepared as previously described (Van Rooijen N, et al. (1994) J Immunol Methods 174:83-93). Six hours before infection with a virus and serum mixture, some groups of naive mice (n=6, BALB/c) were intranasally treated with clodronate liposomes (Roche Diagnostics) to deplete DCs and macrophage cells (Thepen T, et al. (1991) Eur Immunol 21:2845-2850; Bosio C M, et al. (2005) J Immunol 175:6792-6801; McGill J, et al. (2008) J Exp Med 205: 1635-1646).

The depletion efficacy of lung macrophages and DCs was determined by flow cytometry. After 1 d of clodronate liposome treatment, lung tissues were collected and incubated with DNase I (100 μg/mL; Sigma) and type IV collagenase (4 mg/mL; Worthington) for 30 min at 37° C. The single-cell suspensions were stained with fluorescence-conjugated antibodies specific to cell phenotypes (CD11c, CD11b). Cell acquisition was performed with a dual laser flow cytometer (LSR-II; BD Biosciences) and the data were analyzed using FlowJo software (Tree Star).

Results

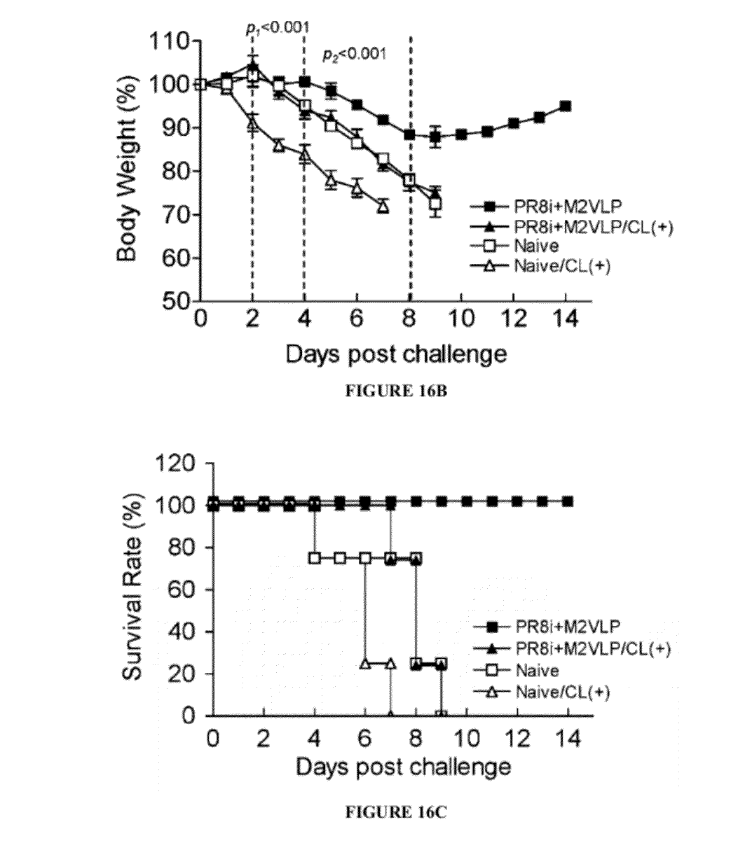

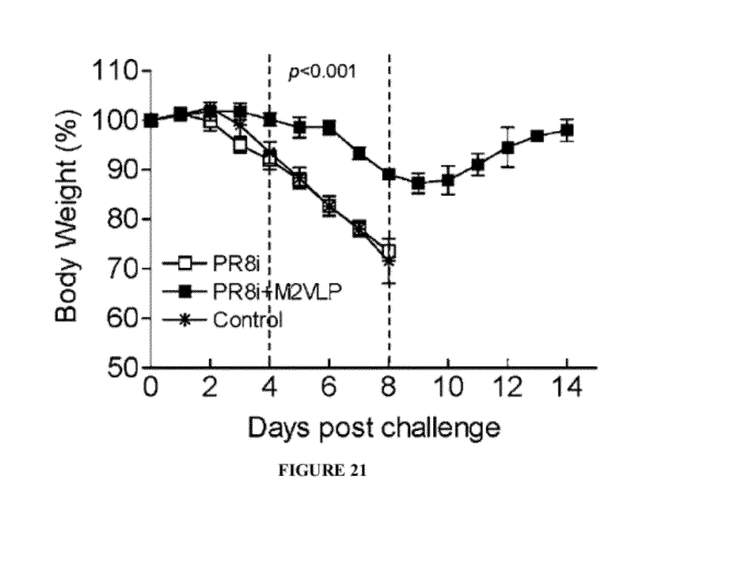

M2 VLP immune sera were tested for their protective efficacy against A/Phil virus (H3N2) in naive mice intranasally infected with a lethal dose of the virus mixed with naive or immune sera. The undiluted sera collected from naive mice or mice immunized with inactivated A/PR8 virus did not confer any protection against lethal infection with H3N2 subtype A/Phil virus (FIG. 16A). In contrast, undiluted or twofold diluted immune sera from the PR8i+M2VLP group conferred complete protection against A/Phil virus without weight loss, and 100% survival was observed even with as much as eightfold diluted immune sera (FIG. 16A). To better understand the potential cross-protective mechanisms, naive mice were pretreated with clodronate liposomes to deplete lung airway dendritic cells (DCs) and macrophage cells. Clodronic acid (INN) or clodronate disodium (USAN) is a bisphosphonate that can selectively deplete macrophages. The depletion efficiency of alveolar DC/macrophage cells was in the range of 60% to 70% at 1 d after treatment, which is similar to the depletion efficiency reported in previous study (McGill J, et al. (2008) Exp Med 205: 1635-46). Cross protection by immune sera from immunization with PR8i+M2 VLP vaccine was not observed in naive mice that were pretreated with clodronate liposomes before infection with mixture of 2009 H1N1 virus and immune sera (PR8i+M2/CL[+]; FIGS. 16B and 16C), although this group showed significant delay in weight loss and mortality compared with naive group treated with clodronate liposomes. Therefore, DC/macrophage immune cells may be important in immune serum-mediated cross protection. Interestingly, mice treated with heat-inactivated immune sera mixed with lethal dose of A/Phil virus showed moderate loss in body weight (FIG. 21), indicating that heat-sensitive serum components such as complement might be involved in providing cross protection.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A cross-protective influenza vaccine comprising a fusion protein immobilized on the surface a virus-like particle (VLP),
wherein influenza virus hemagglutinin (HA) and neuraminidase (NA) are not immobilized on the surface of the VLP;
wherein the fusion protein comprises at least two amino acid sequences comprising an influenza virus matrix protein 2 (M2) extracellular domain; and
wherein the fusion protein optionally comprises at least one adjuvant protein.

2. The vaccine of claim 1, wherein the adjuvant protein is a bacterial flagellin or pilli protein.

3. The vaccine of claim 1, wherein the fusion protein comprises at least four M2 extracellular domains.

4. The vaccine of claim 1, wherein at least one of the M2 extracellular domains comprises the amino acid sequence SEQ ID NO:2.

5. The vaccine of claim 4, wherein the fusion protein comprises the amino acid sequence SEQ ID NO:3.

6. The vaccine of claim 1, wherein the fusion protein comprises at least two M2 extracellular domains from different strains of influenza A.

7. The vaccine of claim 1, wherein the fusion protein comprises a tetramerization domain.

8. The vaccine of claim 7, wherein the tetramerization domain comprises the amino acid sequence SEQ ID NO:4.

9. The vaccine of claim 1, wherein the fusion protein comprises a signal peptide.

10. The vaccine of claim 1, wherein the fusion protein comprises a membrane anchor.

11. The vaccine of claim 1, wherein the membrane anchor comprises a transmembrane domain and optionally a cytoplasmic domain of a viral envelope protein.

12. The vaccine of claim 1, wherein the VLP comprises matrix protein 1 (M1).

13. A method of vaccinating a subject for influenza A comprising administering the vaccine of claim 1 by intranasal, intramuscular, subcutaneous, transdermal or sublingual administration.

14. A method of vaccinating a subject for influenza A comprising
administering to a subject in need thereof a composition comprising inactivated, attenuated influenza A virus or an influenza virus subunit vaccine and a composition comprising a virus-like particle (VLP) assembled from recombinant influenza matrix protein 1 (M1) and matrix protein 2 (M2).

15. The method of claim 14, wherein influenza virus hemagglutinin (HA) and neuraminidase (NA) are not immobilized on the surface of the VLP.

16. The method of claim 14, wherein the VLP is produced by coinfecting insect cells with one or more recombinant baculoviruses expressing the M1 and M2 proteins, culturing the insect cells under physiological conditions, and purifying the VLPs from insect cell culture supernatants.

17. The method of claim 14, wherein the M2 protein comprises full-length M2 protein.

18. The method of claim 14, wherein the M2 protein comprises a fusion protein comprising at least one extracellular domain of an influenza M2.

19. The method of claim 18, wherein the fusion protein comprises at least four extracellular domains of an influenza M2.

20. The method of claim 18, wherein at least one of the M2 extracellular domains comprises the amino acid sequence SEQ ID NO:2.

21. The method of claim 20, wherein the fusion protein comprises the amino acid sequence SEQ ID NO:3.

22. The method of claim 18, wherein the fusion protein comprises at least two M2 extracellular domains from different strains of influenza A.

23. The method of claim 14, wherein the fusion protein comprises an adjuvant protein.

24. The method of claim 23, wherein the adjuvant protein is a bacterial flagellin or pilli protein, or membrane-anchored form of chemokine, TLR agonist, or cytokine.

25. The method of claim 14, wherein the inactivated influenza virus and the VLP are in the same composition.

26. The method of claim 14, wherein the composition comprising inactivated, attenuated influenza A virus or an influenza virus subunit vaccine is administered before or after the composition comprising a virus-like particle VLP.

27. The method of vaccinating of claim 14 comprising administering the vaccine by intranasal, intramuscular, subcutaneous, transdermal or sublingual administration.

28. A composition comprising
inactivated, attenuated influenza A virus or an influenza virus subunit vaccine and a virus-like particle (VLP) assembled from recombinant influenza matrix protein 1 (M1) and matrix protein 2 (M2).

29. The composition of claim 28, wherein the VLP does not comprise influenza virus hemagglutinin (HA) or neuraminidase (NA).

30. The composition of claim 28, wherein the VLP is produced by coinfecting insect cells with one or more recombinant baculoviruses (rBVs) expressing the M1 and M2 proteins, culturing the insect cells under physiological conditions, and purifying the VLPs from insect cell culture supernatants.

31. The composition of claim 28, wherein the M2 protein is full-length M2 protein.

32. The composition of claim 28, wherein the M2 protein is a fusion protein comprising at least one extracellular domain of an influenza M2.

33. The composition of claim 32, wherein the fusion protein comprises multiple copies of extracellular domains of an influenza M2.

34. The composition of claim 32, wherein at least one of the M2 extracellular domains comprises the amino acid sequence SEQ ID NO:2.

35. The composition of claim 34, wherein the fusion protein comprises the amino acid sequence SEQ ID NO:3.

36. The composition of claim 32, wherein the fusion protein comprises at least two M2 extracellular domains from different strains of influenza A.

37. The composition of claim 28, wherein the fusion protein comprises an adjuvant protein.

38. The composition of claim 37, wherein the adjuvant protein is a bacterial flagellin or pilli protein, or membrane-anchored form of chemokine, TLR agonist, or cytokine.

39. The composition of claim 32, wherein the inactivated or attenuated influenza virus and the VLP are in separate containers of a device or kit for use in vaccinating a subject for influenza A.

40. A method of vaccinating a subject for influenza A comprising administering the vaccine of claim 28 by intranasal, intramuscular, subcutaneous, transdermal or sublingual administration.


SEQUENCE LISTING (6 sequences)

SEQ ID NO: 1 - protein, 25 aa, Influenza virus
MSLLTEVETPIRNEWGCRCNDSSDP

SEQ ID NO: 2 - protein, 24 aa, Artificial Sequence
  Modified Extracellular dommain of the Influenza M2 protein
SLLTEVETPIRNEWGSRSNDSSDP

SEQ ID NO: 3 - protein, 135 aa, Artificial Sequence
  Synthetic mofified M2e sequence
VDHMCAAASLLTEVETPIRNEWGSRSNDSSDPAAGTSAAASLLTEVETPIRNEWGSRSNDSSDPAAALQAAASLLTEVETPIRNEWGSRSNDSSDPAAAACAAASLLTEVETPIRNEWGSRSNDSSDPAAAACKL

SEQ ID NO: 4 - protein, 32 aa, Artificial Sequence
  Synthetic M2e construct
ELKQIEDKLEEILSKLYHIENELARIKKLLGE

SEQ ID NO: 5 - protein, 17 aa, Artificial Sequence
  Synthetic modified M2e protein
SLLTEVETPIRNEWGCR

SEQ ID NO: 6 - protein, 27 aa, Human immunodeficiency virus
MQPIQIAIVALVVAIIIAIVVWSIVII